(19) United States
(12) Patent Application Publication
(10) Pub. No.: US 2004/0106166 A1
(43) Pub. Date: Jun. 3, 2004

(54) Enzyme electrode and process for manufacturing the same

(75) Inventor: Toru Matsumoto, Minato-ku (JP)

Correspondence Address: SUGHRUE MION, PLLC, 2100 PENNSYLVANIA AVENUE, N.W., SUITE 800, WASHINGTON, DC, 20037, US

(73) Assignee: NEC CORPORATION

(21) Appl. No.: 10/718,729

(22) Filed: Nov. 24, 2003

Related U.S. Application Data
(63) Continuation of application No. PCT/JP02/07488, filed on Jul. 24, 2002.

(30) Foreign Application Priority Data
Jul. 24, 2001 (JP) 2001-223614
Aug. 3, 2001 (JP) 2001-237180

(51) Int. Cl. C12Q 1/26

(52) U.S. Cl. 435/25; 205/777.5

(57) ABSTRACT

The present invention provides an enzyme electrode exhibiting good measurement performance under wide ranges of the application conditions, being excellent in durability during long-term use and further being producible with a higher yield, as well as a process for manufacturing the enzyme electrode employing a wafer process particularly suitable to mass production. An enzyme electrode according to the present invention comprises an electrode 2 formed on an insulating substrate 1, an immobilized enzyme layer 4 formed over the electrode 2, and a permeation-limiting layer 6 placed on the uppermost surface and over the immobilized enzyme layer 4, wherein on the immobilized enzyme layer 4 is optionally formed an adhesion layer 8 comprising a silane-containing compound, on whose upper surface is formed the permeation-limiting layer 6; or the permeation-limiting layer 6 may be a film mainly comprising a fluorine-containing polymer in which a number of grooves are built on its surface, or alternatively the film has an irregular surface having a surface roughness of 0.0001 or more and 1 or less fold to its average thickness being selected within a range of 0.01 to 1 μm.

### TECHNICAL FIELD

This invention relates to an enzyme electrode and a process for manufacturing the same; in particular, it relates to an enzyme electrode being usable in electrochemical measurement of a particular chemical substance in a solution with use of enzyme reaction thereof and to a biosensor for which it is utilized.

### BACKGROUND ART

A detection technique employing an enzyme reaction in combination with an electrochemical reaction has been extensively used for measuring a variety of components contained in a sample from an organism or the like. For instance, there has been commonly used a biosensor in which a chemical compound in a solution is quantitatively converted into enzyme reaction products and hydrogen peroxide by using the catalytic action of an enzyme, and the resulted hydrogen peroxide is then detected via an oxidation-reduction reaction thereof. For example, in a glucose biosensor, glucose is oxidized by glucose oxidase (GOX) to produce gluconolactone and hydrogen peroxide. Since the amount of the hydrogen peroxide produced thereby is proportional to the level of glucose, the level of glucose in the sample can be quantified by measuring the amount of hydrogen peroxide generated. Catalytic action of an enzyme generally provides reaction products in an amount proportional to a substrate concentration, but there are limitations to a substrate concentration where such proportional relation is kept. Thus, for measuring a substrate in high concentration over the upper limit, a biosensor has permeation-limiting function to reduce the amount of the substrate reaching an enzyme. For example, such an approach that a permeation-limiting layer is formed on an immobilized enzyme layer in an enzyme electrode used in a biosensor has been conventionally applied.

For solving aforementioned problem being left in the conventional technology, we have successfully developed an excellent permeation-limiting layer using a film comprising, as main component thereof, a fluorine-containing polymer having a structure where a pendant group containing at least a fluoro-alkylene block therein is attached to an unfluorinated vinyl-based polymer rather than a film composed of a polymer with high fluorine content such as Teflon®, and we have already applied it for a patent (Japanese Laid-open Patent Publication No. 2000-81409). The enzyme electrode disclosed in Japanese Laid-open Patent Publication No. 2000-81409 has a permeation-limiting layer consisting of said film comprising the polymer having the particular structure as an essential component thereof so that it allows for measurement under a wide variety of application conditions and exhibits good durability to long-term use.

As another example of a biosensor having a permeation-limiting layer, U.S. Pat. No. 5,696,314 has disclosed an enzyme electrode having a porous permeation-limiting layer comprising Teflon® particles or the like, formed on an immobilized enzyme layer. In the enzyme electrode, as shown in FIG. 5, on a substrate 30 is formed an electrode 31 made of platinum or the like, on which is formed an immobilized enzyme layer 32. Then, on the immobilized enzyme layer 32 is formed, via an adhesion layer 33, a polymer layer 34 including the same enzyme as that contained in the immobilized enzyme layer 32. Furthermore, on the polymer layer 34 are formed a permeation-limiting layer 35, an adhesion layer 38 and a protective layer 37. The permeation-limiting layer 35 is a porous film that consists of such essential components as polymer particles, metal particles and a polymer binder. An example using Teflon® (poly-tetrafluoroethylene) as a material for the polymer particles and the polymer binder has been disclosed therein. The permeation-limiting layer 35 is formed by screen printing. Specifically, Teflon binder is first dissolved in a fluorine-containing solvent, and with the solution is kneaded (roll milled) a mixture of alumina and Teflon particles to prepare ink. The ink thus prepared is screen-printed (stenciled) on the polymer layer 34 to form the permeation-limiting layer 35.

However, such a permeation-limiting layer formed using Teflon is lacking in sufficient flexibility, and thus when an adjacent layer swells up, it fails in deforming fully in response to the swelling. Hence, there is a problem to be solved that during using the enzyme electrode, the permeation-limiting layer tends to be detached from an adjacent layer such as the immobilized enzyme layer. Once detachment occurs, there generates a certain gap between the permeation-limiting layer and the surface of a layer such as the immobilized enzyme layer in the enzyme electrode, and there raises a problem that afterwards such a gap makes precise measurement difficult or requires a longer time for removing a liquid soaking into the gap, leading to a longer set-up time for re-measurement.

When using a polymer binder with high fluorine content such as a Teflon binder described in the above patent gazette, it has an inadequate solubility in a solvent so that a solution thereof having a controlled viscosity cannot be prepared. A coating layer, therefore, cannot be formed by a method such as spin coating, and thus it is hard to prepare a permeation-limiting layer in thinner thickness therewith. Additionally, a permeation-limiting layer using a film composed of the polymer with high fluorine content is to be of a porous film to exhibit its controlled permeability, and thus it is necessary for its thickness to be kept somewhat thicker. The above patent gazette has described that the permeation-limiting layer 35 preferably has a thickness of 10 to 40 μm. As described above, there remains a problem that thickness of the permeation-limiting layer must be made thick, which leads to a lower response rate and a longer time for removing a liquid soaking in the permeation-limiting layer after measurement.

Furthermore, as described above, a film composed of a polymer with high fluorine content such as Teflon is lacking in flexibility so that the permeation-limiting layer tends to be broken due to swelling of an adjacent layer thereto. In this respect, it leaves room for improvement. Particularly, in the case where the permeation-limiting layer is placed adjacently to the immobilized enzyme layer being capable of easily swelling, the problem may be significant.

Such a permeation-limiting layer consisting of a film composed of polymer with high fluorine content utilized in an enzyme electrode described in the above patent gazette may not exhibit fully sufficient strength and adhesiveness to an adjacent layer such as an immobilized enzyme layer. Additionally, the permeation-limiting layer consisting of a film made of a polymer with high fluorine content is lacking in flexibility, and thus when an adjacent layer swells up, it fails in deforming fully in response to the swelling. As a result, there is a problem that during using the enzyme electrode, the permeation-limiting layer tends to be detached from the adjacent layer such as the immobilized enzyme layer. Once detachment occurs, there generates a certain gap between the permeation-limiting layer and the surface of such a layer as the immobilized enzyme layer in an enzyme electrode, and there may raise a problem that afterwards such a gap

(i) may make precise measurement difficult or

(ii) may require a longer time for removing a liquid soaking in the enzyme electrode, leading to a longer set-up time for re-measurement.

### DISCLOSURE OF INVENTION

We have conducted intense investigation for large scale production of an enzyme electrode having the structure disclosed in Japanese Laid-open Patent Publication No. 2000-81409, and finally we have found that such an approach as that choice of a thickness of the permeation-limiting layer from the range of 0.01 to 1 μm will improve adhesiveness of the permeation-limiting layer to an underlying layer (for example, an immobilized enzyme layer) is useful for producing an enzyme electrode meeting the designed performance requirement in a higher yield. Since the enzyme electrode having a structure disclosed in Japanese Laid-open Patent Publication No. 2000-81409 comprises the aforementioned permeation-limiting layer using a film comprising, as main component thereof, the fluorine-containing polymer having a particular structure, it exhibits significantly improved adhesiveness to an underlying layer in comparison with a permeation-limiting layer consisting of a film composed of a polymer with high fluorine-content such as Teflon, in which Teflon particles or the like are blended. However, in a process for mass-producing a plurality of enzyme electrodes in a wafer by means of a process for producing a large number of enzyme electrodes on one substrate at the same time, more strong adhesiveness is required between the permeation-limiting layer and its underlying layer (for example, immobilized enzyme layer). During processing a wafer having a multi-layered film comprising an immobilized enzyme layer and a permeation-limiting layer on its surface such as cutting off individual chips from a wafer on which a multi-layered film has been formed and mounting the chips separated alone on a case or the like, said multi-layered film receives a large mechanical load. Therefore, the film desirably has a layered structure possessing good adhesiveness whereby it can endure the load and an adequate deformability.

According to the manufacturing process described in Japanese Laid-open Patent Publication No. 2000-81409, an enzyme electrode exhibiting excellent measurement stability during long-term use can be prepared with good re-productivity as long as the enzyme electrode is produced in the scale of an individual chip process. However, in a process for simultaneously preparing a large number of enzyme electrodes on a single substrate, a so-called wafer process, performance fluctuation in enzyme electrodes tends to be increased in comparison with a process where an enzyme electrode is produced for each chip. In the so-called wafer process, it may be important to investigate a permeation-limiting layer in terms of factors other than film materials constituting the layer, for a mass-producible enzyme electrode having desired performance with a higher yield.

For solving some problems described above in mass production, an aim of the present invention is to provide an enzyme electrode which can be used under a wide variety of application conditions, exhibit good durability in long-term use and give higher productivity. In particular, an aim of the present invention is to provide an enzyme electrode having a structure whereby desired performance can be consistently achieved, even when employing a mass-production process (wafer process).

We have intensely investigated an enzyme electrode structure more suitable for mass production of an enzyme electrode with a higher yield in a wafer process while retaining good properties of the permeation-limiting layer that is obtained by utilizing a film comprising, as main component thereof, a fluorine-containing polymer having a structure where a pendant group containing at least a fluoroalkylene block therein is attached to an unfluorinated vinyl-based polymer disclosed in Japanese Laid-open Patent Publication No. 2000-81409, and also process for manufacturing the same. As a result, we have found that the above problems can be solved by selecting the electrode structure described below when forming the permeation-limiting layer consisting of aforementioned film comprising, as main component thereof, a polymer having the particular structure on the immobilized enzyme layer, and then have brought the present invention to completion.

According to the first aspect of the present invention, the invention provides an enzyme electrode having an electrode structure where an adhesion layer containing a silane-containing compound lies between an immobilized enzyme layer and a permeation-limiting layer. Thus, the enzyme electrode according to the first aspect of the present invention is an enzyme electrode comprising a portion of electrode formed on an insulating substrate; an immobilized enzyme layer formed over the portion of electrode; an adhesion layer containing a silane-containing compound formed over the immobilized enzyme layer; and a permeation-limiting layer comprising a fluorine-containing polymer having a structure where a pendant group containing at least a fluoroalkylene block therein is attached to an unfluorinated vinyl-based polymer, which is formed on the adhesion layer. The first aspect of the present invention also provides a biosensor utilizing the enzyme electrode according to the first aspect of the present invention. In other words, the biosensor according to the first aspect of the present invention is a biosensor comprising an enzyme electrode having the structure defined above.

The enzyme electrode according to the first aspect of the present invention comprises the adhesion layer comprising the silane-containing compound over the immobilized enzyme layer and the permeation-limiting layer that is formed in contact with the upper surface of the adhesion layer and consists of the film comprising the fluorine-containing polymer having structure where the pendant group comprising at least a fluoroalkylene block therein is attached to the unfluorinated vinyl-based polymer. A combination of the film consisting of the fluorine-containing polymer having the particular structure and the adhesion layer may significantly improve adhesiveness between the permeation-limiting layer and its underlying layer (for example, the immobilized enzyme layer), to give a high performance enzyme electrode exhibiting good production stability. The merit of improvement in adhesiveness resulting from the adhesion layer is particularly prominent when the fluorine-containing polymer used in forming the permeation-limiting layer is the aforementioned fluorine-containing polymer having the particular structure. Although the reason or the mechanism is not clearly understood for such improvement in adhesiveness by the adhesion layer comprising said silane-containing compound, it may be supposed that forming the adhesion layer over the immobilized enzyme layer may result in modification of the surface of the underlying layer and thus improve wettability thereto in the fluorine-containing polymer having the particular structure utilized in forming the permeation-limiting layer. For example, when the adhesion layer is made of a silane coupling agent, the silane coupling agent covers the surface of the underlying layer, leading to a lower surface tension, increased surface hydrophilicity, and which is understood to improve wettability of the fluorine-containing polymer having the particular structure.

The effect of improvement in adhesiveness by using the adhesion layer is caused by synergistic effect of said polymer material having the particular structure that the permeation-limiting layer and the silane-containing compound that forms the adhesion layer. Therefore, when using a polymer containing large numbers of fluorine atoms in its main backbone such as Teflon as a polymer material constituting the permeation-limiting layer, such effect of improvement in adhesiveness due to use of said adhesion layer cannot be fully achieved.

In the first aspect of the present invention, it provides the following process as the process for manufacturing the enzyme electrode according to the first aspect of the present invention. Specifically, the process for manufacturing the enzyme electrode according to the first aspect of the present invention is a process for manufacturing an enzyme electrode comprising the steps of:

forming an electrode film on the main surface of an insulating substrate and then patterning the electrode film to form a plurality of portions of electrode;

applying an enzyme-containing liquid to the main surface of the insulating substrate and then drying the insulating substrate to form an immobilized enzyme layer thereon;

forming an adhesion layer comprising a silane-containing compound over the main surface of the insulating substrate;

applying a liquid containing a fluorine-containing polymer having a structure where a pendant group comprising at least a fluoroalkylene block therein is attached to an unfluorinated vinyl-based polymer to the main surface of the insulating substrate and then drying the insulating substrate to form a permeation-limiting layer; and

dicing the insulating substrate to give a plurality of enzyme electrodes.

The process for manufacturing an enzyme electrode according to the first aspect of the present invention is just a process for forming a plurality of enzyme electrode on a single substrate. Conventionally, employed for forming an enzyme electrode is such process where a permeation-limiting layer and so on are formed on a substrate that has been cut into a single chip size in advance. The conventional process will be explained with reference to FIGS. 19 and 20. First, a plurality of portions of electrode are formed on a substrate and then the substrate is cut in chips (FIG. 19(a)). For example, a double-faced tape is applied on the surface of a spinner (FIG. 19(b)), and then a flexible base on which a substrate chip having portions of electrode formed are placed is attached to the spinner via the double-faced tape (FIG. 19(c)). A prescribed solution is dropped on the portions of electrode (FIG. 20(d)), and then the spinner is rotated at a given rate (FIG. 20(e)). The resulting enzyme electrodes are stored at 40° C. in a nitrogen box under nitrogen atmosphere (FIG. 20(f)). In this manufacturing process employing the step of forming such a layer as a permeation-limiting layer in each chip, there is a limit compressing the improvement of production efficiency to forward a plan for mass production. In contrast, the first aspect of the present invention makes use of process comprising the steps of forming a plurality of enzyme electrodes on a substrate and then cutting off the substrate into enzyme electrode chips, and employs an enzyme electrode structure having a permeation-limiting layer comprising the fluorine-containing polymer having the particular structure as a means for ensuring making of enzyme electrodes with a good production stability by said manufacturing process. Such a fluorine-containing polymer having the particular structure is excellent in applicability to an underlying layer and may be prepared as a solution or dispersion with a relatively lower viscosity. Using the properties, for example, a uniform layer may be formed over the whole surface of the substrate by spin coating with improved reproductivity and thereby a plurality of enzyme electrodes may be suitably formed on the substrate.

In said process for manufacturing an enzyme electrode according to the first aspect of the present invention, if the process is performed in such manner where after the step of forming the immobilized enzyme layer, the step of applying a liquid comprising a silane-containing compound to the main surface of the insulating substrate and then drying the insulating substrate to form the adhesion layer is carried out, and followed by the step of applying the fluorine-containing polymer to the upper surface of the adhesion layer coating the main surface of the insulating substrate and then drying the insulating substrate to form the permeation-limiting layer, adhesiveness is much more improved between the permeation-limiting layer and the adhesion layer being underlying, resulting in good production stability. As described above, such good adhesiveness is achieved by synergistic effect of the fluorine-containing polymer material having the particular structure that composes the permeation-limiting layer and the adhesion layer comprising the silane-containing compound.

In a conventional manufacturing process, at the steps of dicing a substrate to provide a plurality of enzyme electrodes or of forming interconnection by bonding to an enzyme electrode, a mechanical stress loaded therein may occasion detachment between the permeation-limiting layer and the underlying layer, or may bring rise to damage of these layers. In the aforementioned process for manufacturing an enzyme electrode according the first aspect of the present invention, as the process has the step of forming the adhesion layer by using a silane-containing compound, prior to the step of forming the permeation-limiting layer, such occurrence of detachment or damage can be effectively prevented during the manufacturing process.

According to the second aspect of the present invention, there is provided an enzyme electrode having a structure comprising a permeation-limiting layer that is formed over an immobilized enzyme layer being placed on the uppermost surface of the enzyme electrode, wherein the surface of the permeation-limiting layer using a fluorine-containing polymer having the aforementioned particular structure has many grooves formed thereon, or alternatively the surface has an irregular shape so as to adjust a surface roughness to an average film thickness within a given range. Specifically, the enzyme electrode according to the second aspect of the present invention is an enzyme electrode comprising:

a portion of electrode formed on an insulating substrate, an immobilized enzyme layer formed on the portion of electrode, and a permeation-limiting layer formed on the immobilized enzyme layer and placed on the uppermost surface;

wherein the permeation-limiting layer consists of a film essentially comprising a fluorine-containing polymer, and many grooves are built in on the surface of the permeation-limiting layer essentially comprising the fluorine-containing polymer. Alternatively, the enzyme electrode according to the second aspect of the present invention is an enzyme electrode comprising

a portion of electrode formed on an insulating substrate, an immobilized enzyme layer formed on the portion of electrode and a permeation-limiting layer formed on the immobilized enzyme layer and placed on the uppermost surface,

wherein the permeation-limiting layer consists of a film essentially comprising a fluorine-containing polymer;

an average thickness of the permeation-limiting layer is selected within a range of 0.01 to 1 μm; and

the surface of the permeation-limiting layer consisting of the film essentially comprising the fluorine-containing polymer has an irregular shape having a surface roughness within a range of 0.0001 or more and 1 or less fold of said average thickness of the permeation-limiting layer.

In the enzyme electrode according to the second aspect of the present invention, the permeation-limiting layer placed on the uppermost surface is also composed of a layer essentially comprising the fluorine-containing polymer having the aforementioned particular structure. Thus, the enzyme electrode according to the second aspect of the present invention is one characterized by such a particular definition for the surface shape of the permeation-limiting layer meeting the requirement. Conventionally, a permeation-limiting layer used in an enzyme electrode has been technically investigated substantially for its materials constituting the permeation-limiting layer and its thickness design, aiming at improving permeation-controlling ability by means of selection of materials and designing its thickness. On the other hand, in the second aspect of the present invention, the surface shape for the permeation-limiting layer placed on the uppermost surface is selected to be a newly designed shape to achieve improvement in long-term measurement stability and measurement precision of the enzyme electrode and a production yield for the enzyme electrode.

As will be specifically demonstrated in Examples later, the enzyme electrode according to the second aspect of the present invention employs a structure comprising, as the permeation-limiting layer, such a layer having a shape with many grooves built in or having an irregular shape on its surface, while having a controlled surface roughness to improve long-term stability and measurement precision in an enzyme electrode and an yield in enzyme electrode production. Although the mechanism for attaining such improvement is not clearly understood, it may be supposed that use of said surface configuration of the permeation-limiting layer may prevent, adhesion of contaminants to the enzyme electrode surface to some extent, the contaminants adhering to such surface of the electrode may be easily removed by washing after measurement, and additionally, the given surface configuration built therein may improve strength of the permeation-limiting layer, all of which may contribute performance improvement for the permeation-limiting layer.

The extent of performance improvement made by shaping grooves in the surface or by controlling its surface roughness within a prescribed range considerably depends on factors such as materials constituting the permeation-limiting layer and its thickness. In the enzyme electrode according to the second aspect of the present invention, when the permeation-limiting layer consist of a layer comprising essentially the aforementioned fluorine-containing polymer having the particular structure and additionally, its thickness is selected within the range defined above, the extent of improvement in the performance of the permeation-limiting layer is significant.

In the second aspect of the present invention, may employ such a method for manufacturing process wherein a multi-layered film comprising the immobilized enzyme layer and the permeation-limiting layer is formed on the surface of wafer; and post to the film formation for the uppermost permeation-limiting layer, the wafer is cut into chips to give enzyme electrodes may be employed to shape grooves in the surface of the permeation-limiting layer placed on the uppermost surface of the enzyme electrode or to adjust a surface roughness of the permeation-limiting layer within the prescribed range. In addition to selection of said method, it is profitable that the process is designed in such a manner where with use of spin coating as method of film formation for the permeation-limiting layer, carried out is the step of applying a liquid comprising the fluorine-containing polymer having the particular structure to the wafer and then drying it to form the permeation-limiting layer, and furthermore the condition for spin coating is set up within those adaptable thereto.

Thus, in the second aspect of the present invention, the following manufacturing process is provided as a novel process for manufacturing the aforementioned enzyme electrode according to the second aspect of the present invention. Specifically, the process for manufacturing an enzyme electrode according to the second aspect of the present invention is a process for manufacturing an enzyme electrode comprising the steps of:

forming an electrode film on the main surface of an insulating substrate and then patterning the electrode film to form a plurality of portions of electrodes;

applying an enzyme-containing liquid to the main surface of the insulating substrate and then drying the insulating substrate to form an immobilized enzyme layer;

applying a liquid containing a fluorine-containing polymer having a structure where a pendant group comprising at least a fluoroalkylene block is attached to an unfluorinated vinyl-based polymer to the main surface of the insulating substrate by spin coating and then drying the insulating substrate to form the permeation-limiting layer; and

dicing the insulating substrate to give a plurality of enzyme electrodes.

In the manufacturing process for an enzyme electrode according to the second aspect of the present invention, as the liquid comprising the fluorine-containing polymer having the particular structure is applied by spin coating and then dried to form the permeation-limiting layer, the permeation-limiting layer in which a number of grooves may be built in on the surface of the permeation-limiting layer formed, or the permeation-limiting layer having a proper surface roughness may be consistently formed thereby.

Additionally, in the manufacturing process for an enzyme electrode according to the second aspect of the present invention, such process constitution may be employed in which after the step of forming the immobilized enzyme layer, the step of applying a liquid comprising a silane-containing compound on the main surface and then drying the insulating substrate to form an adhesion layer; and sequentially, applying the liquid containing said fluorine-containing polymer having the particular structure by spin coating to the upper surface of the adhesion layer and then drying the insulating substrate to form said permeation-limiting layer. In this case, a silane coupling agent is preferably used as said silane-containing compound used for forming the adhesion layer. A combination of the permeation-limiting layer comprising the fluorine-containing polymer having the particular structure and the adhesion layer comprising the silane-containing compound may significantly improve adhesiveness between the permeation-limiting layer and its underlying layer (for example, the immobilized enzyme layer), which gives a high performance enzyme electrode with excellent production stability.

Furthermore, in the second aspect of the present invention, the fluorine-containing polymer having the structure where the pendant group comprising at least a fluoroalkylene block is attached to the unfluorinated vinyl-based polymer thereof may be a fluoroalcohol ester of the polycarboxylic acid (A) in which the polycarboxylic acid (A) is contained as the unfluorinated vinyl-based polymer. Alternatively, it may be a mixture that contains a fluoroalcohol ester of a polycarboxylic acid (A) in which the polycarboxylic acid (A) is contain ed as the unfluorinated vinyl-based polymer and additionally an alkylalcohol ester of a polycarboxylic acid (B). Furthermore, it may be a fluorine-containing polymer composed of a polycarboxylate essentially comprising a fluoroalcohol ester group and an alkylalcohol ester group. With use of such a fluorine-containing polymer having an unfluorinated vinyl-based polymer as a polymer chain backbone thereof, a number of grooves may be built in its surface when employing method for application by spin coating, and thereby a permeation-limiting layer having a proper surface roughness can be formed more consistently.

In the manufacturing process for an enzyme electrode according to the second aspect of the present invention, as for preparation of the liquid comprising the fluorine-containing polymer having the aforementioned particular structure, a solvent comprising a fluorine-containing compound may be used as a solvent therefor. By using the solvent comprising a fluorine-containing compound in the application step by spin coating, a permeation-limiting layer in which a number of grooves are built in on its surface or its surface has a proper surface roughness may be formed further more stably.

The manufacturing process for an enzyme electrode according to the second aspect of the present invention is a process for production of a plurality of enzyme electrodes in scale of wafer. In the prior art, when manufacturing an enzyme electrode, such a method where a permeation-limiting layer and so on are formed on a substrate, which has been cut into chip size in advance, is employed. The conventional method of manufacturing is a manufacturing process using the steps shown in FIGS. 19 and 20, and there is a limit compressing the improvement of production efficiency to forward a plan for mass production. On the other hand, in the second aspect of the present invention, as it makes use of process comprising the steps of forming a plurality of enzyme electrodes on a substrate and then cutting off the substrate into enzyme electrode chips, an applied film with uniformity of an average thickness over the whole surface of the substrate can be formed with good reproductivity by spin coating, and thereby a plurality of enzyme electrodes may be suitably formed on the substrate. Additionally, by employing the process comprising the step of applying a liquid by spin coating and then drying it to form a film, a number of grooves is built in the surface thereof, and thereby a permeation-limiting layer having a proper roughness can be formed over the whole surface of the substrate with a good yield.

### BRIEF DESCRIPTION OF DRAWINGS

FIG. 1 is a cross section view schematically showing an example of a structure of an enzyme electrode according to the first aspect of the present invention.

FIG. 2 is a cross section view schematically showing another example of a structure of an enzyme electrode according to the first aspect of the present invention.

FIG. 3 is a view for illustrating a process for manufacturing an enzyme electrode according to the present invention, and schematically showing arrangement of a number of electrodes for an enzyme electrode chip formed on an insulating substrate 1.

FIG. 4 is a view showing an example of an arrangement of enzyme electrodes according to the present invention.

FIG. 5 is a cross section view schematically showing an example of a configuration of a conventional enzyme electrode.

FIG. 6 is a view schematically showing an example of a biosensor comprising an enzyme electrode according to the present invention.

FIG. 7 is a cross section view schematically showing an example of a structure of an enzyme electrode according to the second aspect of the present invention.

FIG. 8 is a cross section view schematically showing another example of a structure of an enzyme electrode according to the second aspect of the present invention.

FIG. 9 is a drawing showing the results of long-term stability evaluation in a sensor output over passing time for an enzyme electrode of prior art without an adhesion layer 8 described in Example 1.

FIG. 10 is a drawing showing the results of long-term stability evaluation in a sensor output over passing time for an enzyme electrode with an adhesion layer 8 described in Example 1 according to the first aspect of the present invention.

FIG. 11 is a drawing showing the results of evaluation in sensor output change due to ascorbic acid as an interfering substance in a comparative test between an enzyme electrode with the adhesion layer 8 described in Example 2 in accordance with an the first aspect of the present invention and an enzyme electrode of the prior art without the adhesion layer 8 described in Example 2.

FIG. 12 shows graphs (calibration curves) where a sensor output in an enzyme electrode of the prior art without an adhesion layer 8 described in Example 4 is plotted to a glucose concentration; FIG. 12(a) shows individual calibration curves for five enzyme electrodes in total and FIG. 12(b) is a graph where a sensor output average and a standard deviation calculated from sensor outputs of five enzyme electrodes described above are plotted to a glucose concentration.

FIG. 13 shows graphs (calibration curves) where a sensor output in an enzyme electrode with an adhesion layer 8 described in Example 4 according to the first aspect of the present invention is plotted to a glucose concentration; FIG. 13(a) shows individual calibration curves for five enzyme electrodes in total with the adhesion layer 8 thereof being formed using a 0.1 v/v % aqueous solution of γ-aminopropyltriethoxysilane and FIG. 13(b) is a graph where a sensor output average and a standard deviation calculated from sensor outputs of five enzyme electrodes described above are plotted to a glucose concentration.

FIG. 14 shows graphs (calibration curves) where a sensor output in an enzyme electrode with an adhesion layer 8 described in Example 4 according to the first aspect of the present invention is plotted to a glucose concentration; FIG. 14(a) shows individual calibration curves for five enzyme electrodes in total with the adhesion layer 8 thereof being formed using a 0.05 v/v % aqueous solution of γ-aminopropyltriethoxysilane and FIG. 14(b) is a graph where a sensor output average and a standard deviation calculated from sensor outputs of five enzyme electrodes described above are plotted to a glucose concentration.

FIG. 15 shows graphs (calibration curves) where a sensor output in an enzyme electrode with an adhesion layer 8 described in Example 4 according to the first aspect of the present invention is plotted to a glucose concentration; FIG. 15(a) shows individual calibration curves for five enzyme electrodes in total with the adhesion layer 8 thereof being formed using a 0.2 v/v % aqueous solution of γ-aminopropyltriethoxysilane and FIG. 15(b) is a graph where a sensor output average and a standard deviation calculated from sensor outputs of five enzyme electrodes described above are plotted to a glucose concentration.

FIG. 16 shows graphs for seven types of enzyme electrodes with the adhesion layer 8 described in Example 5 according to the first aspect of the present invention, in each of which an adhesion layer 8 is formed using one of different silane coupling agents at a concentration of 0.1 v/v % prepared from a mixed solvent consisting of pure water and ethanol at a final concentration of 5%, where an average sensor output calculated from sensor outputs of five individual enzyme electrodes in total for each type is plotted to a glucose concentration; respectively, the results for enzyme electrodes prepared using the following coupling agents:

s1: (a) γ-aminopropyltriethoxysilane;

s2: (b) γ-aminopropyltrimethoxysilane;

s3: (c) N-phenyl-γ-aminopropyltrimethoxysilane;

s4: (d) γ-chloropropyltrimethoxysilane;

s5: (e) γ-mercaptopropyltrimethoxysilane;

s6: (f) 3-isocyanatopropyltriethoxysilane; and

s7: (g) 3-acryloxypropyltrimethoxysilane.

FIG. 17 shows graphs for four types of enzyme electrodes with the adhesion layer 8 described in Example 6 according to the first aspect of the present invention, in each of which an adhesion layer 8 is formed using γ-aminopropyltriethoxysilane at a concentration of 0.1 v/v % prepared from a mixed solvent consisting of pure water and different organic solvents at a final concentration of 5% or pure water, where an average sensor output calculated from sensor outputs of five individual enzyme electrodes of each type is plotted to a glucose concentration; respectively, the results for enzyme electrodes prepared using the following solvents:

Et: a mixed solvent of pure water and ethanol;

Mt: a mixed solvent of pure water and methanol;

EA: a mixed solvent of pure water and ethyl acetate; and

W: pure water.

FIG. 18 shows graphs for four types of enzyme electrodes according to the first aspect of the present invention, in each of which a permeation-limiting film described in Example 8 is formed using different concentrations of 1H,1H -perfluorooctyl polymethacrylate used in a solution for its film formation, where an average sensor output calculated from sensor outputs of five individual enzyme electrodes of each type is plotted to a glucose concentration.

FIG. 19 shows the steps in a production process according to method for manufacturing an enzyme electrode where the step of forming a coated layer constituting the enzyme electrode is conducted using spin coating for each chip cut from a wafer;

FIG. 19(a) shows the step of mounting of chips cut from a wafer on a flexible base;

FIG. 19(b) shows the step of applying a double-faced tape for mounting said flexible base on a spinner used in spin coating; and

FIG. 19(c) shows the step of mounting said flexible base on the spinner via said double-faced tape.

FIG. 20 shows the steps in a production process according to method for manufacturing an enzyme electrode where the step of forming a coated layer constituting the enzyme electrode is conducted using spin coating for each chip cut from a wafer;

FIG. 20(d) shows the step of droping a coating solution on a chip mounted on a flexible base on a spinner;

FIG. 20(e) shows the step of forming a spin-coat applied film from the droplets of the coating solution droped on the chip by spinner-rotating; and

FIG. 20(f) shows the step of drying said applied film after spin coating to form different types of coating layers.

FIG. 21 shows a graph (calibration curves) where a sensor output in the first enzyme electrode according to the second aspect of the present invention described in Example 10 is plotted to a glucose concentration; specifically individual calibration curves for four enzyme electrodes in total.

FIG. 22 shows a graph (calibration curves) where a sensor output in the second enzyme electrode according to the prior art described in Example 10 is plotted to a glucose concentration; specifically individual calibration curves for four enzyme electrodes in total.

FIG. 23 is a print-out of a three dimensional AFM image for the surface of a permeation-limiting layer 6 in an enzyme electrode sample 1 described in Example 9 with the permeation-limiting layer 6 formed by spin coating, placed on the uppermost surface, according to the second aspect of the present invention.

FIG. 24 is a histogram showing a surface roughness distribution determined based on the AFM image for the surface of the permeation-limiting layer 6 shown in FIG. 23, as described in Example 9.

FIG. 25 is a print-out of a two dimensional AFM image where irregularity is indicated by gradation step in which a concave (groove) is indicated as white, observed for the surface of a permeation-limiting layer 6 in an enzyme electrode described in Example 9 with the permeation-limiting layer 6 formed by spin coating, placed on the uppermost surface, according to the second aspect of the present invention.

### BEST MODE FOR CARRYING OUT THE INVENTION

The first and the second aspects of the present invention will be more particularly explained with reference to embodiments specific for each aspect.

First, the first to forth embodiments will be described as a preferred embodiment of the first aspect of the present invention.

### The First Embodiment

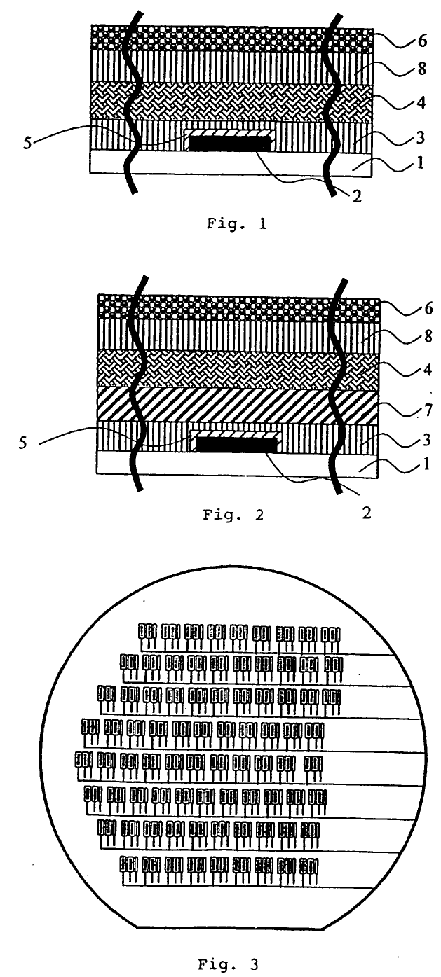

The first embodiment according to the first aspect of the present invention will be described with reference to the drawings. FIG. 1 shows the configuration of an enzyme electrode according to the first embodiment. As shown in FIG. 1, an electrode 2 as a working electrode is formed on an insulating substrate 1 and an electrode protective layer 5 essentially consisting of an urea compound is formed covering the upper surface of the electrode. The electrode protective layer 5 is selectively formed in the portion for the electrode 2. On these layers is formed a binding layer 3 mainly made of γ-aminopropyltriethoxysilane, on which is then formed an immobilized enzyme layer 4 where an enzyme has been immobilized in an organic polymer as a matrix. On the layer is formed an adhesion layer 8 made of γ-aminopropyltriethoxysilane. On the upper surface of the adhesion layer 8 is then formed a permeation-limiting layer 6 comprising a fluoroalcohol ester of a polycarboxylic acid resin as main component thereof.

As for substance for the insulating substrate 1, substances essentially consisting of a highly-insulative material such as ceramics, glass, quartz and plastics are applicable. A material used for the insulating substrate 1 is preferably selected form materials being excellent in waterproof, heat resistance, chemical resistance and adhesiveness to an electrode.

For instance, a conductive material comprising such as platinum may be used as a material for the electrode 2, gold, silver and carbon as main component therein; among others, particularly preferable is platinum, which is excellent in chemical resistance and performance for detection of hydrogen peroxide. The electrode 2 on the insulating substrate 1 may be formed by such method as spattering, ion plating, vacuum deposition, chemical vapor deposition and electrolysis; among others, preferred is use of sputtering. With use of sputtering, good adhesiveness between the conductive material film formed and the insulating substrate 1 is attained and a platinum layer can be easily formed. Furthermore, with the purpose of improving adhesiveness of the electrode 2 to the insulating substrate 1, such a layer as titanium or chromium layer may be inserted therebetween.

The electrode protective layer 5 covering the electrode 2 limits permeation into the electrode of contaminant such as urea, which is contained in a sample for measurement. For example, the electrode protective layer 5 may be composed of a urea compound. Examples of a urea compound include,) but not limited to, urea, thiourea or the like, and among others, preferably used is urea with low toxicity and low cost. The enzyme electrode of the present invention has a structure in which the electrode protective layer comprising a contaminant such as a urea compound is formed on the surface of the electrode in advance, which will prevent occurrence of fluctuation in sensitivity that results from contamination due to permeation into the surface of the electrode of contaminants such as urea. Thus, in the light of such function of the electrode protective layer, it will be apparent that variety of urea compounds usable in the electrode protective layer are not limited to the kinds exemplified above.

The electrode protective layer 5 may be formed by such method as immersion, plasma polymerization and electrolysis; among others, preferable is electrolysis, which can be conducted in a shorter process time by using an inexpensive apparatus. Specifically, it is preferable that the insulating substrate on which the electrode has been formed is immersed in a mixed solution containing a supporting electrolyte and a urea compound, and electric current is applied t o form the electrode protective layer thereon. When using urea as the urea compound therefor, a urea concentration in the mixed solution is preferably selected from the range of 0.1 mM to 6.7 M, more preferably of 1 M to 6.7 M. When using sodium chloride as the supporting electrolyte therewith, a sodium-chloride concentration in the mixed solution is preferably selected from the range of 0.1 mM to 2 M, more preferably of 1.5 mM to 150 mM. Selecting the film-forming conditions described above may provide an electrode protective layer with high quality, which may effectively suppress adhesion of contaminants and may prevent permeation of an interfering substance to a reaction with hydrogen peroxide in the electrode 2 to achieve good selectivity to the reaction with hydrogen peroxide in the electrode 2. Furthermore, equipping the electrode protective layer 5 may improve adhesiveness to the binding layer 3 formed thereon.

The binding layer 3 formed on the electrode protective layer 5 may improve adhesiveness (binding strength) of the immobilized enzyme layer 4 formed thereon to the insulating substrate 1 and the electrode protective layer 5. The binding layer 3 may also improve wettability of the surface of the insulating substrate 1 and may have the effect of improving thickness uniformity of the immobilized enzyme layer 4 when forming thereon the immobilized enzyme layer 4 in which an enzyme is immobilized. Additionally, the binding layer 3 exhibits selective permeation to ascorbic acid, uric acid and acetaminophen, which may interfere with a reaction of hydrogen peroxide on the electrode 2. For example, the binding layer 3 may be made of a silane coupling agent. Examples of a silane coupling agent usable thereto include

vinyltrichlorosilane, vinyltrimethoxysilane, vinyltriethoxysilane,

β-(3,4-epoxycyclohexyl)ethyltrimethoxysilane,

γ-glycidoxypropyltrimethoxysilane,

γ-glycidoxypropylmethyldiethoxysilane,

γ-glycidoxypropyltriethoxysilane,

γ-methacryloxypropylmethyldimethoxysilane,

γ-methacryloxypropyltrimethoxysilane,

γ-methacryloxypropylmethyldiethoxysilane,

γ-methacryloxypropyltriethoxysilane,

N-(β-aminoethyl)-γ-aminopropylmethyldimethoxysilane,

N-(β-aminoethyl)-γ-aminopropyltrimethoxysilane,

N-(β-aminoethyl)-γ-aminopropyltriethoxysilane,

γ-aminopropyltrimethoxysilane,

γ-aminopropyltriethoxysilane,

N-phenyl-γ-aminopropyltrimethoxysilane,

γ-chloropropyltrimethoxysilane,

γ-sulfanylpropyltrimethoxysilane,

3-isocyanatopropyltriethoxysilane,

3-acryloxypropyltrimethoxysilane, and

3-triethoxysilyl-N-(1,3-dimethyl-butylidene)propylamine; and among others, preferably used is γ-aminopropyltriethoxysilane that is of an aminosilane type, in the light of an interlayer binding force and selective permeability. For instance, the binding layer 3 may be formed by spin coating with a silane coupling agent solution. When forming the binding layer 3 by spin coating with the silane coupling agent solution, the concentration of the silane coupling agent is preferably about 1 v/v % (% by volume) so that selective permeability can be significantly improved with the choice of such conditions.

The immobilized enzyme layer 4 is a layer in which a catalytic enzyme is immobilized using an organic polymer as matrix (binder) therefor. The immobilized enzyme layer 4 may be formed on the binding layer 3, for example, by dropping and then spin coating with a solution containing some kind of enzyme, a protein cross-linking agent such as glutaraldehyde and albumin. Albumin may protect variety of the enzymes from a reaction with the cross-linking agent and may fill the role of a base for the enzyme protein. Enzymes suitable therefor include enzymes being capable of generating hydrogen peroxide as a product of their catalytic reaction by the enzymes, or consuming oxygen therefor, which is selected from oxidases such as lactate oxidase, glucose oxidase, urate oxidase, galactose oxidase, lactose oxidase, sucrose oxidase, ethanol oxidase, methanol oxidase, starch oxidase, amino acid oxidase, monoamine oxidase, cholesterol oxidase, choline oxidase and pyruvate oxidase.

In an immobilized enzyme layer 4, two or more enzymes may be used in combination for generating hydrogen peroxide. An example fitting with the combinational use of two or more enzymes is set of creatininase, creatinase and sarcosine oxidase, use of which enzymes allows creatinine to be detected. In the immobilized enzyme layer 4, an enzyme may be combined with a coenzyme thereof. An example fitting with the combinational use of the enzyme and coenzyme therewith is pair of 3-hydroxylactate dehydrogenase and nicotinamide adeninedinucleotide (NAD), use of which enzyme and coenzyme allows 3-hydroxylactic acid to be detected. In the immobilized enzyme layer 4, an enzyme may be combined with an electron mediator, where the electron mediator that has been reduced by the enzyme is oxidized on the electrode surface and a current being generated thereby is then measured by th e electrode 2. For instance, an example fitting with use of the enzyme associated with the electron mediator is glucose oxidase with potassium ferricyanide, use of which in the enzymatic reaction system allows glucose to be detected.

As described above, there are no limitations to the structure of the immobilized enzyme layer 4 as long as it comprises at least an enzyme and has function for acting on a target substance for detection to convert it into an electrode sensitive substance such as hydrogen peroxide.

There are no limitations to a method for forming the immobilized enzyme layer 4 as long as it is a method capable of forming a uniform layer; and such as spin coating, spray coating and dipping may be used. Among others, preferably used is spin coating, use of which allows consistent formation of an immobilized enzyme layer having homogeneous quality and thickness.

The adhesion layer 8 formed on the immobilized enzyme layer 4 has function for improving adhesiveness between the immobilized enzyme layer 4 and the permeation-limiting layer 6 formed thereon. In a conventional enzyme electrode, at the steps of dicing a substrate to provide a plurality of enzyme electrodes, or of forming interconnection by bonding to an enzyme electrode, there are some occasions of detachment between the permeation-limiting layer and the underlying layer, or damage within these layers. In contrast, in an enzyme electrode according the first aspect of the present invention, the adhesion layer is formed using a silane-containing compound before forming the permeation-limiting layer, so that such detachment can be effectively prevented thereby. As the result, the enzyme electrodes with uniform properties can be manufactured using a so-called wafer process. Additionally, when forming the permeation-limiting layer 6, it is effective in improvement of thickness homogeneity or surface flatness in the permeation-limiting layer formed thereon. Furthermore, comparing with interfering substances such as ascorbic acid, uric acid and acetaminophen interfering the reaction with hydrogen peroxide on the electrode 2, selective permeability in the permeation-limiting layer for a target substance for detection may be improved into excellent level.

For example, the adhesion layer 8 may be made of a silane coupling agent. Variety of silane coupling agents being usable for the adhesion layer may include series of compounds listed above as examples of silane coupling agent applicable for the binding layer 3. In the adhesion layer 8, an aminosilane, particularly γ-aminopropyltriethoxysilane may be also preferably used in the light of such properties as adhesiveness threreof. The use of 3-isocyanatopropyltriethoxysilane or 3-acryloxypropyltrimethoxysilane may be also effective.

As method for applying a coupling agent solution or the like for the adhesion layer 8 or the binder layer 3, such method as spin coating, spraying, dipping and hot-gas flowing may be used. Spin coating refers to a method where a solution or dispersion of a component for an adhesion layer or binding layer such as a coupling agent is applied using a spin coater. With use of spin coating method, the binding layer or adhesion layer with a thinner thickness may be formed with good thickness controlling. Spraying refers to a method where such liquid as a coupling agent solution atomized is blew up by spray on the substrate surface, and dipping refers to a method where a substrate is immersed in such liquid as a coupling agent solution. Using these methods for formation of a coating film by application, a binding layer or adhesion layer may be formed through a simple procedure without use of any special apparatus. On the other hand, hot-gas flowing refers to a method where a substrate is placed under hot atmosphere and vapor of a coupling agent is flown thereon. By the hot-gas flowing, a binding layer or adhesion layer with a thin thickness may be also formed with good thickness controlling.

When the adhesion layer 8 is made of a coupling age nt, among others, spin coating of a silane coupling agent solution is preferably used. Use of the adhesion layer made of a silane coupling agent formed by spin coating may provide consistent achievement of good adhesiveness. In application by the spin coating, a concentration of the silane coupling agent in the solution is preferably selected from the range of 0.01 to 5 v/v %, more preferably of 0.05 to 1 v/v %. As for a solvent for the silane coupling agent solution, pure water; alcohols such as methanol, ethanol and isopropyl alcohol; and esters such as ethyl acetate may be used alone or in combination of two or more thereof. Among others, mixtures diluted with pure water of ethanol, methanol or ethyl acetate are preferable. In the adhesion layer made of a silane coupling agent, which is formed by spin coating with use of said mixtures of solvent, improvement in adhesiveness thereby is particularly significant. Besides, the adhesion layer 8 has also effect on significant improvement in selective permeability by the permeation-limiting layer.

After application of the coupling agent solution or the like, drying the applied film containing the solvent is carried out. A temperature for drying is generally, but not limited to, within range of room temperature (25° C.) to 170° C. A duration for drying depends on the drying temperature, but is generally within 0.5 to 24 hours. Drying may be conducted in the air or also in an inert gas such as nitrogen. For example, nitrogen blowing, in which nitrogen gas is blown on a substrate to dry up, may be employed.

As for composing material for the permeation-limiting layer 6, a polymer in which a pendant group comprising at least a fluoroalkylene block is attached to an unfluorinated vinyl-based polymer is used. With use of such a polymer comprising the unfluorinated vinyl-based polymer, as polymer backbone thereof, to which the pendant group containing at least the fluoroalkylene block is attached, its adhesiveness to the adhesion layer as an underlying layer is significantly improved. The “unfluorinated vinyl-based polymer” therein is a moiety having function involved in its good adhesiveness to another organic polymer layer such as an immobilized enzyme layer. In contrast, when using such a polymer that has large numbers of fluorine contained in a polymer part other than the pendant group, its adhesiveness to another organic polymer layer such as the immobilized enzyme layer may be reduced, and furthermore, it may be hard to prepare a solution containing said polymer and thus to form the permeation-limiting layer as a thin film thereof. Said unfluorinated vinyl-based polymer is a polymer having a main backbone consisting of carbon-carbon bonds; preferable examples thereof include a homopolymer or copolymer of one or more monomers selected from the group consisting of unsaturated hydrocarbons, unsaturated carboxylic acids and unsaturated alcohols. Among these unfluorinated vinyl-based polymers, a polymer of polycarboxylic acid type is particularly preferable. In such choice of the polymer type, its adhesiveness to the adhesion layer as an underlying layer may be more significantly improved, and thus a permeation-limiting layer with good durability may be provided thereby. In addition, as the pendant group to the vinyl-based polymer, it is preferable that the fluoroalkylene block is attached via an ester group. An ester group has proper polarity, a permeation-limiting layer mainly comprising a polymer in which at least a fluoroalkylene block is attached via an ester group as a pendant group to the vinyl-based polymer can exhibit significant adhesiveness to an underlying layer coated by the adhesion layer. Besides, a pendant group containing a fluoroalkylene block refers to a pendant group comprising a fluoroalkylene as a component unit thereof. On the other hand, a fluoroalkylene refers to an alkylene group, part or all of whose hydrogen atoms are replaced with fluorine atoms.

As described above, the composing materials for the permeation-limiting layer 6 comprises the polymer in which the pendant group comprising at least the fluoroalkylene block is attached to the unfluorinated vinyl-based polymer, and among others, a fluoroalcohol ester of a polycarboxylic acid is particularly preferable. Examples of said polycarboxylic acid include polyacrylic acid, polymethacrylic acid and a copolymer of acrylic acid and methacrylic acid. Herein, the fluoroalcohol ester of the polycarboxylic acid refers to that in which part or all of plurality of carboxy groups present in the polycarboxylic acid are esterified with the fluoroalcohol. The fluoroalcohol refers to that in which all or at least one of hydrogen atoms in the alcohol are replaced with fluorine atoms. All of the carboxy groups present in the polycarboxylic acid may be esterified, or at least part of those may be esterified. To achieve uniform properties, it is desirable that 0.1% or more of the plurality of carboxy groups present in the polycarboxylic acid is esterified. The number of carbons contained in the polyfluoroalcohol is preferably in range of C5 to C9 in the light of good durability after film deposition, more preferably C8 in the light of facility in film deposition. On the other hand, as for the position of OH in the polyfluoroalcohol, preferable is of primary alcohol type, use of which provides best performance for its durability and chemical resistance. In the fluoroalcohol esters of the polycarboxylic acid, particularly preferable are 1H,1H-perfluorooctyl polymethacrylate and 1H,1H,2H,2H-perfluorodecyl polyacrylate. Use of said fluoroalcohol esters of the polycarboxylic acid may achieve good permeation control, facilitate film deposition and exhibit high resistance to an acid, an alkali and a variety of organic solvents.

In addition to the fluoroalcohol ester of the polycarboxylic acid, an alkylalcohol ester of a polycarboxylic acid may be introduced as a component in material for the permeation-limiting layer. For example, the permeation-limiting layer may be made of a mixture of a fluoroalcohol ester of a polycarboxylic acid (A) and an alkylalcohol ester of a polycarboxylic acid (B), or the permeation-limiting layer may be mainly made of an ester compound of polycarboxylic acid with structure having a fluoroalcohol ester and an alkylalcohol ester groups therein. The polycarboxylic acid (A) and the polycarboxylic acid (B) comprised in the aforementioned mixture may be the same or different. The phrase “mainly comprising” means that the above polymer is a main component composing the permeation-limiting layer; for example, the content of the polymer is 50 wt % or more in the permeation-limiting layer. When the permeation-limiting layer is formed into such a composition comprising the aforementioned polymer as main component, an enzyme electrode exhibiting good high temperature stability is obtained therewith. Furthermore, a molecular weight (average molecular weight) of the polymer constituting the permeation-limiting layer is preferably within range of 1000 to 50000, more preferably of 3000 to 30000. An excessively larger molecular weight may make it difficult to prepare a solution containing said polymer and to form a thinner permeation-limiting layer thereby. When an excessively lower molecular weight is used, there may be occasions that adequate permeation control is not achieved in the resulted permeation-limiting layer. Herein, a molecular weight is an average molecular weight over number, which can be determined by GPC (Gel Permeation Chromatography).

The permeation-limiting layer 6 may be formed by spin-coating with a solution containing the aforementioned fluorine-containing polymer on the upper surface of the adhesion layer 8 that is underlying thereof. After forming the adhesion layer 8 has been formed in advance on the immobilized enzyme layer 4 in which a catalytically active enzyme has been immobilized, the permeation-limiting layer 6 may be formed by dropping and then spin coating with a solution of a polyfluoroalcohol ester of a polymethacrylic acid diluted in a perfluorocarbon solvent such as perfluorohexane. When using the spin coating, a content of the fluorine-containing polymer in the solution is preferably adjusted to 0.1 to 5 wt %, more preferably about 0.3 wt %, depending on a target substance for detection. Use of the solution in such a concentration range for formation a film by spin coating may provide achievement of better permeation control in the permeation-limiting layer 6 obtained. Besides, as for method for forming the permeation-limiting layer 6, any method, for example, spin coating, spray coating and dipping may be used without limitation as long as it provides a uniform film thickness. Among others, preferably used is spin coating, as described above. When film forming by spin coating, a permeation-limiting layer with uniform quality and thickness may be obtained consistently thereby. On the other hand, a suitable thickness for the permeation-limiting layer 6 is preferably within 0.01 to 3 μm, more preferably 0.01 to 1 μm. By using a permeation-limiting layer 6 having such a thickness, improvement in an response rate of the enzyme electrode as well as reduced time for washing after measurement can be attained.

By forming a permeation-limiting layer 6 comprising, as main component, a fluorine-containing polymer having the aforementioned particular structure, adhesion of contaminants such as proteins and urea compounds to an enzyme electrode can be prevented. Thus, an electrode protective layer 5 as shown in FIG. 1 may be formed, which can provide synergistical effect for preventive effect due to the electrode protective layer 5 on adhesion of contaminants in addition to the effect of the permeation-limiting layer 6, and also such effect that stable performance in output properties are attained during long term use thereby. A combination of the underlying adhesion layer 8 with the permeation-limiting layer 6 may give good permeation control, which can provide such effect that range of measurement concentration is significantly widened therein. Furthermore, as adhesiveness between the permeation-limiting layer 6 and the adhesion layer 8 is heightened, the detachment may occur rarely, which allows stable measurement of a target substance in a solution during a long term. Moreover, in manufacturing steps for mass production thereof using a wafer process, the multi-layered structure may be little damaged during the steps post to the step of forming the multi-layered film, resulting in a higher yield.

In the first aspect of the present invention, when using a sensor having the enzyme electrode according to the first embodiment as a glucose sensor, the outermost permeation-limiting layer 6 restricts a diffusion rate of glucose, and in the immobilized enzyme layer 4 using a glucose oxidase, glucose diffusing in generates hydrogen peroxide and gluconolactone therefrom by catalytic reaction by oxygen. Of the two, an oxidation current when hydrogen peroxide resulted reaches the electrode 2 is measured to determine the glucose concentration being contained in the sample. As for an electrode system during measurement, an external conventional reference electrode is used in the case of two-electrode method, while both of a counter and a reference electrodes are simultaneously immersed in a solution to be measured in the case of three-electrode method.

### The Second Embodiment

In FIG. 2, illustrated is a configuration of an enzyme electrode of the second embodiment according to the first aspect of the present invention. In the enzyme electrode shown in FIG. 2, on an insulating substrate 1 is formed an electrode 2 operating as a working electrode, and an electrode protective layer 5 mainly comprising an urea compound is formed to cover upper surface thereof. Over these is formed a binding layer 3 mainly consisting of γ-aminopropyltriethoxysilane, on which are sequentially formed an ion-exchange resin layer 7 made of a perfluorocarbon sulfonic acid resin, an immobilized enzyme layer 4 in which an enzyme is immobilized by using an organic polymer as matrix therefor, an adhesion layer 8 formed with γ-aminopropyltriethoxysilane, and finally, on the adhesion layer 8, a permeation-limiting layer 6 comprising a fluoroalcohol ester of a polycarboxylic acid resin as main component thereof.

The electrode 2, electrode protective layer 5, binding layer 3, immobilized enzyme layer 4, adhesion layer 8 and permeation-limiting layer 6 formed on the insulating substrate 1 can be sequentially formed in similar manner as described in said first embodiment.

In the enzyme electrode of the second embodiment, as a perfluorocarbon sulfonic acid resin such as Nafion (trade name) may be used fro composing an ion-exchange resin layer 7. Nafion is a commercially available cation-exchange resin, which has a structure where a perfluoropolyalkylene ether side chain having a sulfonic acid end group is attached to a perfluoromethylene main chain.

By arranging the ion-exchange resin layer 7 such as a Nafion film under the immobilized enzyme layer 4, influence of interfering substances to the electrode 2 can be eliminated. Thus, its effect works synergistically with an effect of limiting permeation of interfering substance to the electrode 2 by the binding layer 3, the adhesion layer 8 and the electrode protective layer 5, which may significantly reduce influence of the interfering substances on measurement precision for the enzyme electrode of the second embodiment.

The ion-exchange resin layer 7 is formed by dropping and spin coating such a solution of Nafion that is prepared by solving with 50% ethanol in pure water on the binding layer 3 consisting of a γ-aminopropyltriethoxysilane layer. Examples of a solvent for said Nafion solution used in spin coating include alcohols such as isopropyl alcohol and ethyl alcohol. A Nafion concentration in a dropped solution is preferably 1 to 10 w/v %, more preferably 5 to 7 w/v %. By forming by spin coating using a solution with a concentration within such range, effect of the ion-exchange resin layer 7 obtained therewith for eliminating influence of the interfering substances on the electrode 2 may be significant.

### The Third Embodiment

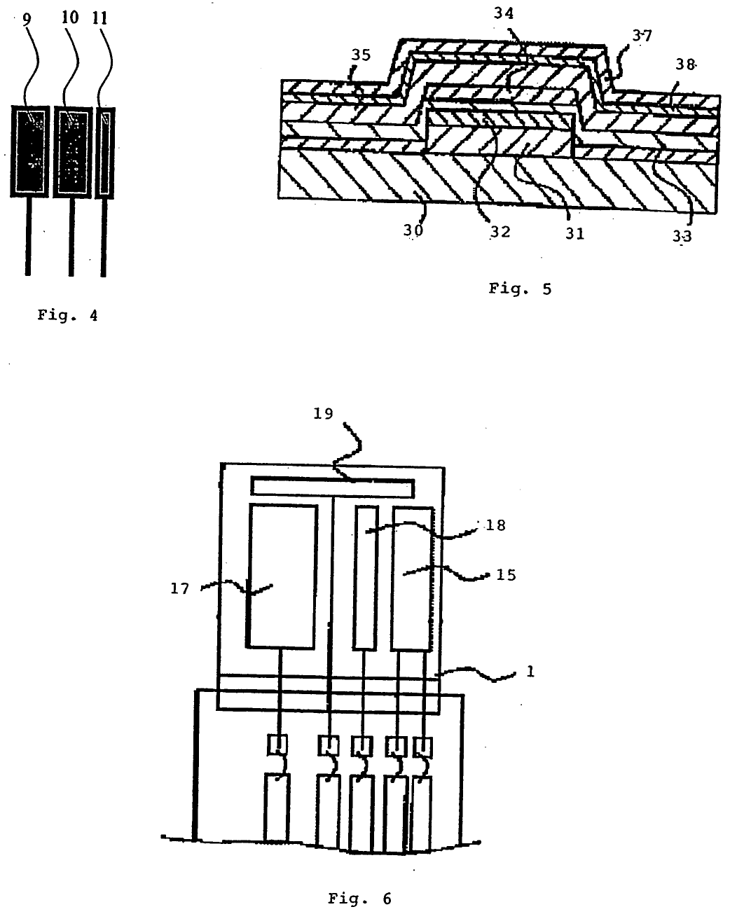

A manufacturing process for an enzyme electrode according to the first aspect of the present invention will be described with reference to FIGS. 3 and 4. In the manufacturing process explained as the third embodiment, on a wafer 12 made of an insulating material is first formed an electrode film, which is then patterned to form a plurality of working electrodes 9, counter electrodes 10 and reference electrodes 11, respectively. In FIG. 3, displayed is the situation at the end of step of forming the electrodes. Then, on the wafer 12 is applied an enzyme-containing solution by such method as spin coating, and then the wafer 12 is dried to form an immobilized enzyme layer at least over the working electrode 9.

Then, a solution containing the silane-containing compound described above is applied on the wafer 12 being finished with the step of forming the immobilized enzyme layer, and then the wafer 12 is dried to form an adhesion layer.

Then, on the upper surface of the adhesion layer is applied a solution of the fluorine-containing polymer having the aforementioned particular structure, and the wafer 12 is dried to form a permeation-limiting layer. Using the fluorine-containing polymer such as a fluoroalcohol ester of a polycarboxylic acid as a component composing the permeation-limiting layer, the thin permeation-limiting layer with uniform quality and thickness may be consistently formed. In addition, when using such a fluorine-containing polymer material, a viscosity of a solution containing the fluorine-containing polymer can be set in low so that a thin permeation-limiting layer can be consistently formed by spin coating.

After that, the wafer 12 is diced to provide a plurality of enzyme electrodes. In FIG. 4, illustrated is a configuration of an enzyme electrode for the three-electrode method. The enzyme electrode for the three-electrode method has a configuration where a working electrode 9, a counter electrode 10 and a reference electrode 11 are placed in a single chip. The working electrode 9 and the counter electrode 10 may be similar to that as described for the electrode 2 in the first and the second embodiments. A material used for the reference electrode 11 is preferably silver/silver chloride.

The configuration shown in FIG. 4, in which the working, the counter and the reference electrodes are formed on the single insulating substrate, allows a solution to be replaced during operation of a sensor. That is, as long as the sensor surface is wetted by, for example, an electrolyte, the working, the counter and the reference electrodes are electrically connected to each other, and therefore, even when the sensor is temporarily in contact with the air during replacement of the solution, measurement can be continued without any trouble. Furthermore, it allows for precise electrochemical measurement by the three-electrode method; in particular, an enzyme electrode for detecting a fine current can be realized.

### The Fourth Embodiment

In FIG. 6, shown is a structure of a biosensor using an enzyme electrode according to the first aspect of the present invention. In the biosensor demonstrated as the fourth embodiment, on an insulating substrate 1 are arranged a working electrode 17, a counter electrode 18, and a reference electrode 19, and further is formed a temperature sensor 15 therewith. The surfaces of the working electrode 17, the counter electrode 18 and the reference electrode 19 are individually covered by a multi-layered film that has the layered structure shown in FIG. 1.

In the fourth embodiment, one type of working electrode is employed in the enzyme electrode used in the biosensor, but a sensor structure comprising a plurality of working electrodes in which different immobilized enzyme layers are formed may be employed. Furthermore, in addition to the temperature sensor, such configuration in which another sensor such as a pH sensor may be also placed may be acceptable. On the other hand, the working electrode 17, the counter electrode 18 and the reference electrode 19 constituting the enzyme electrode for the three-electrode method may be appropriately arranged. Moreover, in the fourth embodiment, there has been described the biosensor consisting of three electrodes, i. e., the working, the counter and the reference electrodes, but alternatively, a biosensor itself may have a configuration where a working electrode made of platinum and a reference electrode may be formed on a quartz substrate.

In the fourth embodiment, an amperometric type sensor has been illustrated, but an enzyme electrode according to the first aspect of the present invention may be also, of course, applied to an ion-sensitive field-effect transistor type of sensor.

Furthermore, the fifth to the seventh embodiments will be described as a preferable embodiment of the second aspect of the present invention.

The Fifth Embodiment

The fifth embodiment according to the second aspect of the present invention will be explained with reference to the drawings. In FIG. 7, demonstrated is a configuration of an enzyme electrode of the fifth embodiment. As shown in FIG. 7, in the enzyme electrode of the fifth embodiment, on an insulating substrate 1 is formed an electrode 2 operating as a working electrode, on which are sequentially formed a binding layer 3 mainly comprising γ-aminopropyltriethoxysilane, and further an immobilized enzyme layer 4 in which an enzyme has been immobilized with an organic polymer as matrix therefor, and finally, on immobilized enzyme layer 4, a permeation-limiting layer 6 comprising a fluoroalcohol ester of a polycarboxylic acid resin, as main component therefor. There are built-in many grooves in the surface of the permeation-limiting layer 6 placed on the uppermost surface.

In the enzyme electrode according to the second aspect of the present invention, applicable for the insulating substrate 1 and the electrode 2 thereof may be those similar as described for the insulating substrate and the electrode constituting the enzyme electrode according to the first aspect of the present invention. Furthermore, preferred embodiments for the insulating substrate and the electrode are the same to preferred embodiments described above for the enzyme electrode according to the first aspect of the present invention.

Used for the binding layer 3 formed on the electrode 2 may be also that similar as described for the binding layer formed on the electrode protective layer in the enzyme electrode according to the first aspect of the present invention. In such case, a preferred embodiment for the binding layer in the enzyme electrode of the fifth embodiment is also the same to the preferred embodiment described above for the binding layer in the enzyme electrode according to the first aspect of the present invention. In the enzyme electrode of said fifth embodiment, the binding layer 3 can improve adhesiveness (binding force) of the immobilized enzyme layer 4 formed thereon with the insulating substrate 1 as well as the electrode 2. The binding layer 3 may improve wettability of the surface of the insulating substrate 1 so that uniformity in a thickness of the immobilized enzyme layer 4 may be improved when forming the immobilized enzyme layer 4 in which an enzyme has been immobilized. Furthermore, the binding layer 3 exhibits selective permeability to ascorbic acid, ureic acid or acetaminophen capable of interfering the reaction of hydrogen peroxide in the electrode 2.

In the enzyme electrode according to the second aspect of the present invention, used for the immobilized enzyme layer 4 may be similar one as that described above for the immobilized enzyme layer used in the enzyme electrode according to the first aspect of the present invention. Herein, a preferred embodiment of the immobilized enzyme layer is also the same to the preferred embodiment mentioned above in the enzyme electrode according to the first aspect of the present invention.

In the enzyme electrode according to the second aspect of the present invention, as for the permeation-limiting layer 6 placed on the uppermost surface, the permeation-limiting layer 6 comprising the fluorine-containing polymer that has specific surface forms where many grooves are built in on the surface of the permeation-limiting layer 6 is used, so that adhesion of contaminants such as proteins and urea compounds to the enzyme electrode may be prevented. Thus, owing to such preventing effect to adhesion of contaminants by the permeation-limiting layer 6, the effect that allows consistent output properties to be given even during long-term use will be achieved. Since the permeation-limiting layer 6 having the particular surface configuration is placed on the uppermost surface, good permeation control can be achieved thereby, and such effect that range of a measured concentration may be significantly widened may be provided. In the second aspect of the present invention, for example, when using a sensor comprising the enzyme electrode of the fifth embodiment as a glucose sensor, the permeation-limiting layer 6 being arranged on the uppermost surface restricts a diffusion rate of glucose, and in the immobilized enzyme layer 4 using a glucose oxidase, glucose diffusing in generates hydrogen peroxide and gluconolactone as a result of a catalytic reaction by oxygen. Of the two, an oxidation current when hydrogen peroxide reaches the electrode 2 is measured to determine a glucose concentration being contained in a sample. As for an electrode system during measurement, an external conventional reference electrode is used in the case of two-electrode method, while both of a counter and a reference electrodes therein are simultaneously immersed in a solution for detection in the case of three-electrode method.

Therefore, in the enzyme electrode according to the second aspect of the present invention, the surface configuration of the permeation-limiting layer 6 on the uppermost surface is formed to meet the following (i), (ii) or both.

(i) a number of grooves are built-in on the surface the permeation-limiting layer.

(ii) An average thickness of the permeation-limiting layer is selected to be in the range of 0.01 to 1 μm, preferably 0.02 to 0.5 μm, and a surface roughness of the permeation-limiting layer is 0.0001 or more and 1 or less fold of the average thickness of the permeation-limiting layer, preferably 0.001 or more and 1 or less fold thereof.

Thus, by selecting such a constitution that permeation-limiting layer 6 having the surface with a number of grooves built in thereon, or with an irregular shape showing said surface roughness is placed on the uppermost surface, the enzyme electrode according to the second aspect of the present invention is designed as an enzyme electrode that is usable under wider range of application conditions, good in durability during long-term use and excellent in higher productivity. Furthermore, when using a manufacturing process utilizing a wafer process for mass production, it may be used as an enzyme electrode having a structure which can consistently give desired performance. Although the mechanism of these effects to be attained is not clearly understood, the constitution with use of the permeation-limiting layer 6 having the surface structure described above is selected, which prevents adhesion of contaminants to the enzyme electrode surface to some extent, and by forming the particular surface configuration, strength of the permeation-limiting layer is improved, which is supposed to contribute to the improvement in performance.

There are no particular restrictions to the size of the plurality of grooves built in on the surface of the permeation-limiting layer 6, but they preferably have a small size being observable by an electron microscope, in particular, an atomic force microscope having excellent analytic performance in three dimensional directions. More specifically, depths of the grooves may be selected in the range of 0.1 to 100 nm, more preferably 0.5 to 30 nm.

For building in many grooves on the surface of the permeation-limiting layers or adjusting surface roughness of the permeation-limiting layer to a given range, it is effective to employ a manufacturing process where a multi-layered film comprising an immobilized enzyme layer and a permeation-limiting layer on a wafer surface and after film deposition, the wafer is cut into chips to give enzyme electrodes; and to use spin coating as a film formation method for the permeation-limiting layer in the step of forming the permeation-limiting layer which is conducted for a wafer, and further to adjust the spin-coating conditions adequately therefor. For example, the steps for forming a multi-layered film, which are conducted for a wafer, comprise at least the steps of forming an electrode film on the main surface of an insulating substrate and then patterning the electrode film to form a plurality of portions of electrode 2; applying a solution containing an enzyme on the main surface of the insulating substrate and then drying the insulating substrate to form an immobilized enzyme layer 4; and applying a solution containing a fluorine-containing polymer having a structure where a pendant group comprising at least a fluoroalkylene block is attached to an unfluorinated vinyl-based polymer by spin coating and then drying the insulating substrate to form a permeation-limiting layer 6. Then, a manufacturing process for an enzyme electrode may used, in which the step for dicing the insulating substrate being finished with said formation of the multi-layered film to provide a plurality of enzyme electrodes is carried out at the end, which allows formation of the permeation-limiting layer having the particular surface structure described above with good production stability.

The followings are preferable as the conditions in spin coating the solution comprising the fluorine-containing polymer on a wafer surface for said step of forming the permeation-limiting layer 6. A rotation speed for a spinner used is preferably 500 rpm or more, more preferably 2000 rpm or more. The upper limit of the spinner rotation rate is, for example, 600 rpm or less, even though depending on a thickness of an applied film. A temperature during the application of film is preferably 0° C. or more and 40° C. or less, for example, application of film may be suitably conducted about 4° C. The choice of the applied material is expected to significantly influence a shape, in particular a thickness or surface shape, of a permeation-limiting layer to be formed, and will be later detailed.

The permeation-limiting layer 6 used for the enzyme electrode according to the second aspect of the present invention is made of a fluorine-containing polymer. As is for the first aspect of the present invention, as for a preferable polymer material mainly composing the permeation-limiting layer 6, exemplified is a polymer in which a pendant group having at least a fluoroalkylene block is attached to an unfluorinated vinyl-based polymer. By using said fluorine-containing polymer having the particular structure as a main component, the surface of the permeation-limiting layer formed can be reliably controlled to a suitable shape, and thus a desired groove or irregularity shape can be attained. As a result, it may improve measurement stability in the enzyme electrode according to the second aspect of the present invention and a yield thereof in the production process.

In addition, in the enzyme electrode the second aspect of the present invention, use of such a polymer having a backbone of an unfluorinated vinyl-based polymer to which at least a pendant group having a fluoroalkylene block is attached as component composing the permeation-limiting layer 6 may remarkably improve its adhesiveness to the underlying layer. For such a purpose, in the enzyme electrode according to the second aspect of the present invention, as component composing the permeation-limiting layer 6, used similarly may be those illustrated above as material composing for the permeation-limiting layer in the enzyme electrode according to the first aspect of the present invention. Additionally, as for the composing components for the permeation-limiting layer 6, preferable embodiments may be the same to the preferable embodiments described above for the enzyme electrode according to the first aspect of the present invention.

In the enzyme electrode of the fifth embodiment shown in FIG. 7 according to the second aspect of the present invention, the permeation-limiting layer 6 may be formed by spin-coating with the solution containing the aforementioned fluorine-containing polymer on the upper surface of the underlying immobilized enzyme layer 4. The permeation-limiting layer 6 may be formed by dropping a solution of a polyfluoroalcohol ester of a polymethacrylic acid diluted with a perfluorocarbon solvent such as perfluorohexane on the immobilized enzyme layer 4 in which a catalytically active enzyme has been immobilized, followed by spin coating of the solution for application. When using the method of spin coating, a content of said fluorine-containing polymer in the solution is preferably adjusted to 0.1 to 5 wt %, more preferably about 0.3 wt %, depending on a target substance to be measured. By forming a film by spin coating using a solution at a concentration in such a range, better permeation control may be achieved in the permeation-limiting layer 6 obtained. Besides, as for technique for forming the permeation-limiting layer 6, any method such as spin coating, spray coating and dipping may be used without limitation as long as it provides a uniform film thickness. Among others, when using a wafer process, use of spin coating is preferable as explained above. When the formation of film is made by spin coating, a permeation-limiting layer having uniform quality and thickness may be obtained consistently thereby. On the other hand, when employing the configuration (i) where a number of grooves are built in on the surface of the permeation-limiting layer, a thickness of the permeation-limiting layer 6 is preferably 0.01 to 1 μm, more preferably 0.02 to 0.5 μm, further preferably 0.04 to 0.25 μm. By using a permeation-limiting layer 6 having such a thickness, an improvement in a response rate of the enzyme electrode and reduction in time for washing post to measurement may be attained.

### The Sixth Embodiment

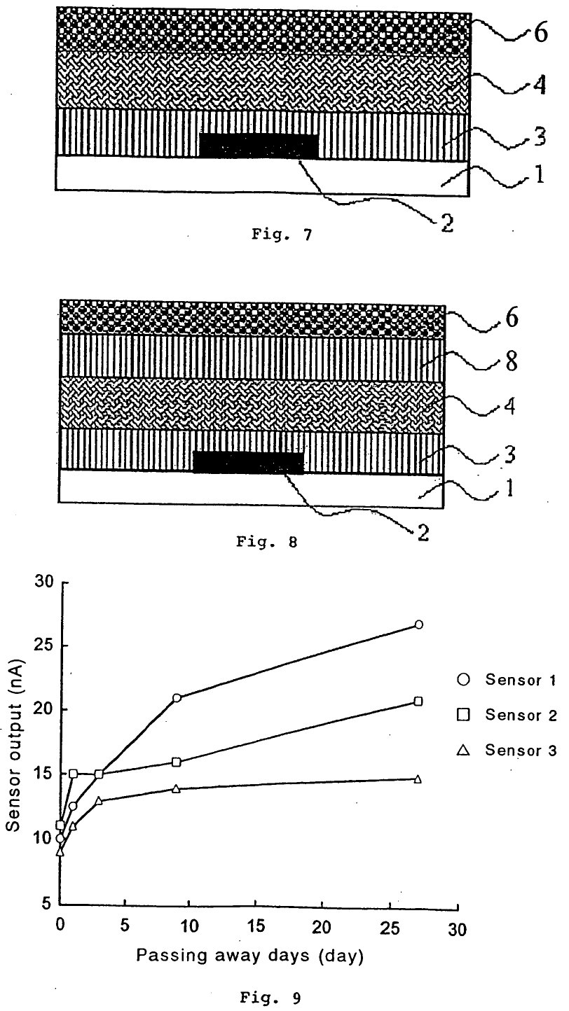

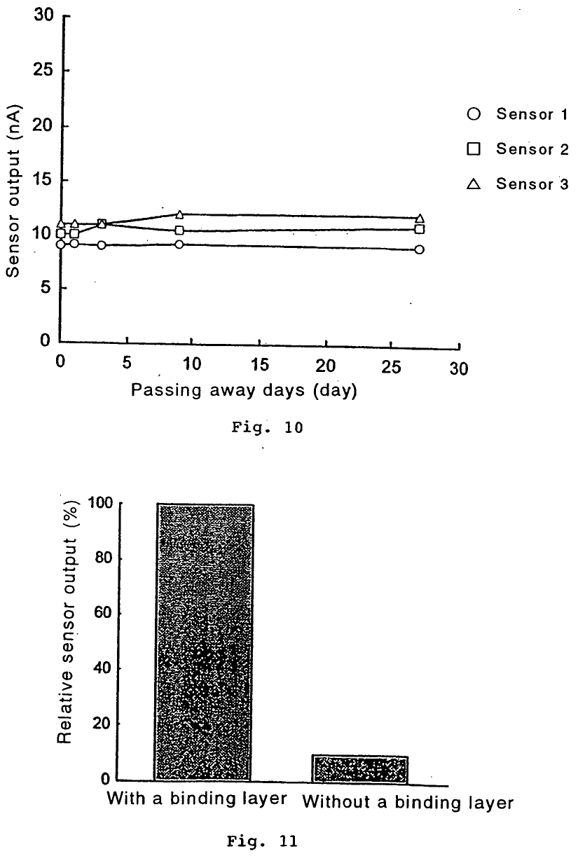

In FIG. 8, shown is a configuration of an enzyme electrode of the sixth embodiment according to the second aspect of the present invention. In the enzyme electrode shown in FIG. 8, on an insulating substrate 1 is formed an electrode 2 functioning as a working electrode, on which is sequentially formed a binding layer 3 mainly made of γ-aminopropyltriethoxysilane, an immobilized enzyme layer 4 in which an enzyme has been immobilized with an organic polymer as matrix therefor, and an adhesion layer 8 mainly made of γ-aminopropyltriethoxysilane, and finally on the adhesion layer 8, a permeation-limiting layer 6 comprising a fluoroalcohol ester of a polycarboxylic acid resin as main component.

The electrode 2, the binding layer 3, the immobilized enzyme layer 4 and the permeation-limiting layer 6 formed on the insulating substrate 1 are sequentially formed in similar manner as described for the fifth embodiment according to the second aspect of the present invention.

As is for the adhesion layer used in the aforementioned enzyme electrode according to the first aspect of the present invention, the adhesion layer 8 formed on the immobilized enzyme layer 4 plays a role to improve adhesiveness between the immobilized enzyme layer 4 and the permeation-limiting layer 6 formed thereon in the enzyme electrode of the sixth embodiment. Accordingly, the adhesion layer 8 used for the enzyme electrode of the sixth embodiment is preferably similar one as described above for the adhesion layer used in the enzyme electrode according to the first aspect of the present invention.

Therefore, in the enzyme electrode of the sixth embodiment, the adhesion layer 8 may be also made of, for example, a silane coupling agent such as γ-aminopropyltriethoxysilane as described above in the binding layer 3. Additionally, as for the adhesion layer in the enzyme electrode of the sixth embodiment, a preferable embodiment may be the same to the preferable embodiment described above for the adhesion layer in the enzyme electrode according to the first aspect of the present invention.

Furthermore, in the second aspect of the present invention, used as a method for application of such a solution of coupling agent for the adhesion layer 8 and the binding layer 3 may be application methods used for forming the adhesion layer and the binding layer in the first aspect of the present invention. Among others, when making adhesion layer 8 of a coupling agent in the second aspect of the present invention, spin coating of a silane coupling agent solution is preferably used as in the first aspect of the present invention. Furthermore, as for the step of forming the adhesion layer and the conditions therein for of the enzyme electrode of the sixth embodiment, its preferred embodiments are also the same to the preferred embodiments described above for the adhesion layer of the enzyme electrode according to the first aspect of the present invention.

### The Seventh Embodiment

In FIG. 6, illustrated is a structure of a biosensor using an enzyme electrode according to the second aspect of the present invention. In the biosensor shown as the seventh embodiment, on an insulating substrate 1 are also arranged a working electrode 17, a counter electrode 18, and a reference electrode 19, and further is formed a temperature sensor 15 therewith. The surfaces of the working electrode 17, the counter electrode 18 and the reference electrode 19 are individually covered by a multi-layered film that has the layered structure, as shown in FIG. 7.

In the seventh embodiment, one type of working electrode is employed in the enzyme electrode used in the biosensor, but a sensor structure comprising a plurality of working electrodes in which different immobilized enzyme layers are formed may be employed. Furthermore, in addition to the temperature sensor, such configuration in which another sensor such as a pH sensor may be also placed may be acceptable. On the other hand, the working electrode 17, the counter electrode 18 and the reference electrode 19 constituting the enzyme electrode for the three-electrode method may be appropriately arranged. Moreover, in the seventh embodiment, there has been described the biosensor consisting of three electrodes, i. e., the working, the counter and the reference electrodes, but alternatively, a biosensor itself may have a configuration where a working electrode made of platinum and a reference electrode may be formed on a quartz substrate.

In the seventh embodiment, an amperometric type sensor has been illustrated, but an enzyme electrode according to the second aspect of the present invention may be also, of course, applied to an ion-sensitive field-effect transistor type of sensor.

### EXAMPLES

This invention will be more specifically described with reference to Examples. 1H,1H-perfluorooctyl polymethacrylate used In these examples is Florard FC-722 available from Sumitomo-3M with an average molecular weight Mn of about 6000 to 8000 (GPC measurement value).

Examples 1 to 8 presented below represent the most preferred embodiments according to the first aspect of the present invention, but the first aspect of the present invention is not limited to these specific examples.

### Example 1

As shown in FIG. 3, on a 4-inch quartz wafer 12 (thickness: 0.515 mm; Nippon Electric Glass Co., Ltd.) were formed 82 sets of electrode chip, each set of which had the configuration shown in FIG. 4 and comprised a working electrode 9 (area: 5 mm²), a counter electrode 10 (area: 5 mm²) made of platinum, and a reference electrode 11 (area: 1 mm²) made of silver/silver chloride. When cutting into the individual sets, the size of each electrode chip is 10 mm×6 mm. Then, the chip was immersed in a 6M solution of urea containing 150 mM sodium chloride, and 0.7 V was applied to the working electrode 9 in relation to the reference electrode 11 for 10 min. In practice, all the working electrodes 9 were interconnected as shown in FIG. 3 and connected to the periphery. Thus, the periphery and the reference electrode 11 were connected to an electrochemical measuring apparatus, and the above potential was applied. Thus, an urea layer as an electrode protective layer 2 was formed on the working electrode 9 by electrolysis.

Then, a 1 v/v % aqueous solution of γ-aminopropyltriethoxysilane (hereinafter, referred to as “APTES” as appropriate) was spin-coated to form a binding layer 3. Then, a 22.5 w/v % solution of albumin containing glucose oxidase and 1 v/v % glutaraldehyde was spin-coated to form an immobilized enzyme layer 4. Then, a 0.1 v/v % aqueous solution of γ-aminopropyltriethoxysilane was spin-coated to form an adhesion layer 8. Subsequently, a 0.3 wt % 1H,1H-perfluorooctyl polymethacrylate solution prepared using perfluorohexane as a solvent was spin-coated to form a permeation-limiting layer 6 made of 1H,1H-perfluorooctyl polymethacrylate. Thus, an enzyme electrode wafer was prepared.

Finally, the wafer was diced with a glass scribing apparatus to provide enzyme electrodes. Three of the enzyme electrode chips prepared were appropriately selected. Each chip was connected to a flexible substrate via a wire bonding and then the connecting part was waterproofed.

As a control, an enzyme electrode wafer was prepared as described above, except that an adhesion layer 8 was not formed between the immobilized enzyme layer 4 and the permeation-limiting layer 6. Again, three of the enzyme electrode chips prepared were appropriately selected, and each chip was connected to a flexible substate via a wire bonding and then the connecting part was waterproofed.

The enzyme electrodes thus prepared were stored by immersing them in a pH7 buffer of TES (N-tris(hydroxymethyl)methyl-2-aminoethanesulfonic acid) containing 150 mM sodium chloride. For a 200 mg/dl glucose solution containing the TES buffer, a current as a sensor output was measured at Days 0, 1, 3, 9 and 27, and stability of the measured sensor output was evaluated. A storing temperature was 24° C. and a potential was not applied during storage.

As the evaluation results, FIG. 9 shows variation over time of a sensor output for the enzyme electrode without an adhesion layer 8 between the immobilized enzyme layer 4 and the permeation-limiting layer 6, whereas FIG. 10 shows variation over time of a sensor output for the enzyme electrode having an adhesion layer 8 between the immobilized enzyme layer 4 and the permeation-limiting layer 6. Comparison of these results demonstrate that the adhesion layer 8 formed between the immobilized enzyme layer 4 and the permeation-limiting layer 6 can provide a stable sensor output for a long time and variation in a sensor output can be minimized.

### Example 2

As shown in FIG. 3, on a 4-inch quartz wafer 12 (thickness: 0.515 mm; Nippon Electric Glass Co., Ltd.) were formed 82 sets of electrode chip, each set of which had the configuration shown in FIG. 4 and comprised a working electrode 9 (area: 5 mm²), a counter electrode 10 (area: 5 mm²) made of platinum and a reference electrode 11 (area: 1 mm²) made of silver/silver chloride. When cutting into the individual sets, the size of each electrode chip is 10 mm×6 mm. Then, the chip was immersed in a 6M solution of urea containing 150 mM sodium chloride, and 0.7 V was applied to the working electrode 9 in relation to the reference electrode 11 for 10 min. In practice, all the working electrodes 9 were interconnected as shown in FIG. 3 and connected to the periphery. Thus, the periphery and the reference electrode 11 were connected to an electrochemical measuring apparatus, and the above potential was applied. Thus, an urea layer as an electrode protective layer 2 was formed on the working electrode 9 by electrolysis.

Then, a 1 v/v % aqueous solution of γ-aminopropyltriethoxysilane was spin-coated to form a binding layer 3. Then, a 5 w/v % solution of a perfluorocarbonsulfonic acid resin was spin-coated to form an ion-exchange resin layer 7 mainly made of the perfluorocarbonsulfonic acid resin (Nafion) on the binding layer 3. Then, a 22.5 w/v % solution of albumin containing glucose oxidase and 1 v/v % glutaraldehyde was spin-coated to form an immobilized enzyme layer 4. Then, a 0.1 v/v % aqueous solution of γ-aminopropyltriethoxysilane was spin-coated to form an adhesion layer 8. Subsequently, a 0.3 wt % 1H,1H-perfluorooctyl polymethacrylate solution prepared using perfluorohexane as a solvent was spin-coated to form a permeation-limiting layer 6 made of 1H,1H-perfluorooctyl polymethacrylate. Thus, an enzyme electrode wafer was prepared.

Finally, the wafer was diced with a glass scribing apparatus to provide enzyme electrodes. Twenty of the enzyme electrode chips prepared were appropriately selected. Each chip was connected to a flexible substrate via a wire bonding and then the connecting part was waterproofed.

As a control, an enzyme electrode wafer was prepared as described above, except that an adhesion layer 8 was not formed between the immobilized enzyme layer 4 and the permeation-limiting layer 6. Again, twenty of the enzyme electrode chips prepared were appropriately selected, and each chip was connected to a flexible substrate via a wire bonding and then the connecting part was waterproofed.

The enzyme electrodes thus prepared were stored by immersing them in a pH7 buffer of TES (N-tris(hydroxymethyl)methyl-2-aminoethanesulfonic acid) containing 150 mM sodium chloride. For a 200 mg/dl ascorbic acid solution containing the TES buffer, a current as a sensor output was measured and influence of ascorbic acid to the measured sensor output was evaluated. A storing temperature was 24° C. and a potential was not applied during storage.

As the evaluation results, the sensor outputs from twenty enzyme electrodes were averaged and FIG. 11 shows a sensor output from the enzyme electrode having the adhesion layer 8 between the immobilized enzyme layer 4 and the permeation-limiting layer 6 as a relative value to a sensor output from the enzyme electrode without an adhesion layer 8 between the immobilized enzyme layer 4 and the permeation-limiting layer 6 as 100%. Comparison of these results demonstrate that the adhesion layer 8 formed between the immobilized enzyme layer 4 and the permeation-limiting layer 6 can reduce influence of ascorbic acid as an interfering substance to 1/10.

### Example 3

As shown in FIG. 3, on a 4-inch quartz wafer 12 (thickness: 0.515 mm; Nippon Electric Glass Co., Ltd.) were formed 82 sets of electrode chip, each set of which had the configuration shown in FIG. 4 and comprised a working electrode 9 (area: 5 mm²), a counter electrode 10 (area: 5 mm²) made of platinum and a reference electrode 11 (area: 1 mm²) made of silver/silver chloride. When cutting into the individual sets, the size of each electrode chip is 10 mm×6 mm. Then, the chip was immersed in a 6M solution of urea containing 150 mM sodium chloride, and 0.7 V was applied to the working electrode 9 in relation to the reference electrode 11 for 10 min. In practice, all the working electrodes 9 were interconnected as shown in FIG. 3 and connected to the periphery. Thus, the periphery and the reference electrode 11 were connected to an electrochemical measuring apparatus, and the above potential was applied. Thus, an urea layer as an electrode protective layer 2 was formed on the working electrode 9 by electrolysis.

Then, a 1 v/v % aqueous solution of γ-aminopropyltriethoxysilane was spin-coated to form a binding layer 3. Then, a 5 w/v % solution of a perfluorocarbonsulfonic acid resin was spin-coated to form an ion-exchange resin layer 7 mainly made of the perfluorocarbonsulfonic acid resin (Nafion) on the binding layer 3. Then, a 22.5 w/v % solution of albumin containing glucose oxidase and 1 v/v % glutaraldehyde was spin-coated to form an immobilized enzyme layer 4. Then, a 0.1 v/v % aqueous solution of γ-aminopropyltriethoxysilane was spin-coated to form an adhesion layer 8. Subsequently, a 0.3 wt % 1H,1H-perfluorooctyl polymethacrylate solution prepared using perfluorohexane as a solvent was spin-coated to form a permeation-limiting layer 6 made of 1H,1H-perfluorooctyl polymethacrylate. Thus, an enzyme electrode wafer was prepared.

Finally, the wafer was diced with a glass scribing apparatus to provide enzyme electrodes. One enzyme electrode chip was randomly selected and was connected to a flexible substrate via a wire bonding and then the connecting part was waterproofed.

As a control, an enzyme electrode wafer was prepared as described above, except that an adhesion layer 8 was not formed between the immobilized enzyme layer 4 and the permeation-limiting layer 6. Again, one enzyme electrode chip was randomly selected and was connected to a flexible substrate via a wire bonding and then the connecting part was waterproofed.

The enzyme electrodes thus prepared were stored by immersing them in a pH7 buffer of TES (N-tris(hydroxymethyl)methyl-2-aminoethanesulfonic acid) containing 150 mM sodium chloride. Ten repeated and consecutive measurements were conducted for a normal urine control from BioRad Inc. (Lifocheck) containing about 20 mg/dl glucose. From the measured values from the above two enzyme electrodes, a standard deviation was calculated for evaluating repetition reproductivity. As the evaluation results, Table 1 shows repetition reproductivity as a relative value in relation to an average measured value as a reference.

TABLE 1

| | Repetition |
|---|---|
| | reproductivity |
| With an adhesion layer | 2.5% |
| Without an adhesion | 3.1% |
| layer | |

Comparing these results, the enzyme electrode with the adhesion layer 8 between the immobilized enzyme layer 4 and the permeation-limiting layer 6 gave a repetition reproductivity of 2.5%, while the enzyme electrode without the adhesion layer 8 between the immobilized enzyme layer 4 and the permeation-limiting layer 6 gave 3.1%, showing that the enzyme electrode with the adhesion layer 8 between the immobilized enzyme layer 4 and the permeation-limiting layer 6 was better.

### Example 4

As shown in FIG. 3, on a 4-inch quartz wafer 12 (thickness: 0.515 mm; Nippon Electric Glass Co., Ltd.) were formed 82 sets of electrode chip, each set of which had the configuration shown in FIG. 4 and comprised a working electrode 9 (area: 5 mm²), a counter electrode 10 (area: 5 mm²) made of platinum and a reference electrode 11 (area: 1 mm²) made of silver/silver chloride. When cutting in to the individual sets, the size of each electrode chip is 10 mm×6 mm. Then, the chip was immersed in a 6M solution of urea containing 150 mM sodium chloride, and 0.7 V was applied to the working electrode 9 in relation to the reference electrode 11 for 10 min. In practice, all the working electrodes 9 were interconnected as shown in FIG. 3 and connected to the periphery. Thus, the periphery and the reference electrode 11 were connected to an electrochemical measuring apparatus, and the above potential was applied. Thus, an urea layer as an electrode protective layer 2 was formed on the working electrode 9 by electrolysis.

Then, a 1 v/v % aqueous solution of γ-aminopropyltriethoxysilane was spin-coated and dried at 40° C. for 1 hour under nitrogen atmosphere to form a binding layer 3. Then, a 5 w/v % solution of Nafion was spin-coated and dried at 40° C. for 1 hour under nitrogen atmosphere to form an ion-exchange resin layer 7 mainly made of Nafion on the binding layer 3. Then, a 22.5 w/v % solution of albumin containing glucose oxidase and 1 v/v % glutaraldehyde was spin-coated to form an immobilized enzyme layer 4.

Then, three aqueous solutions of γ-aminopropyltriethoxysilane at concentrations of 0.05 v/v %, 0.1 v/v % and 0.2 v/v % using pure water as a solvent were spin-coated and dried at 40° C. for 1 hour under nitrogen atmosphere to form three adhesion layers 8 with different average film thickness. In addition, a wafer without an adhesion layer 8 was prepared as a control.

Subsequently, a 0.3 wt % 1H,1H-perfluorooctyl polymethacrylate solution prepared using perfluorohexane as a solvent was spin-coated to form a permeation-limiting layer 6 made of 1H,1H-perfluorooctyl polymethacrylate on four wafers described above to prepare enzyme electrode wafers.

Finally, the wafer was diced with a glass scribing apparatus to provide enzyme electrodes. Five of the enzyme electrode chips prepared were appropriately selected. Each chip was connected to a flexible substrate via a wire bonding and then the connecting part was waterproofed.

The four types of enzyme electrodes thus prepared were stored by immersing them in a pH7 buffer of TES (N-tris(hydroxymethyl)methyl-2-aminoethanesulfonic acid) containing 150 mM sodium chloride. For a solution of glucose at 0 to 2000 mg/dl containing the TES buffer, a current as a sensor output was measured and a calibration curve was plotted for each of the five enzyme electrodes. Furthermore, for each of the four types of enzyme electrodes, an average and a standard deviation in the calibration curve for the five enzyme electrodes were calculated.

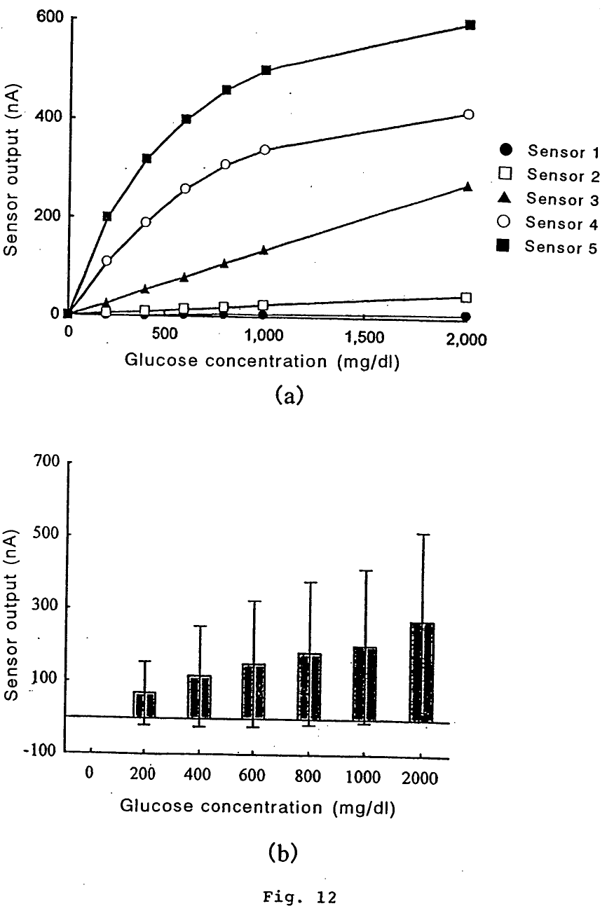

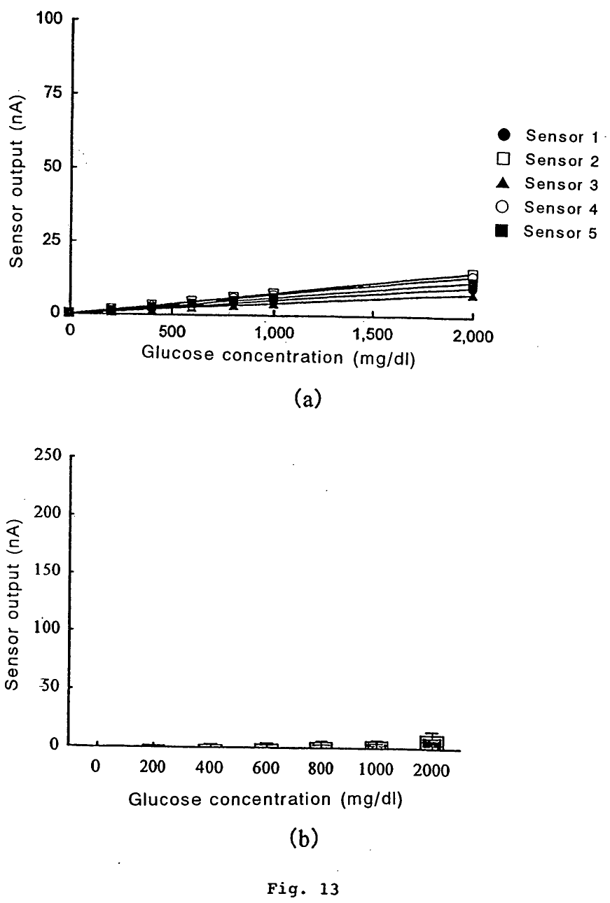

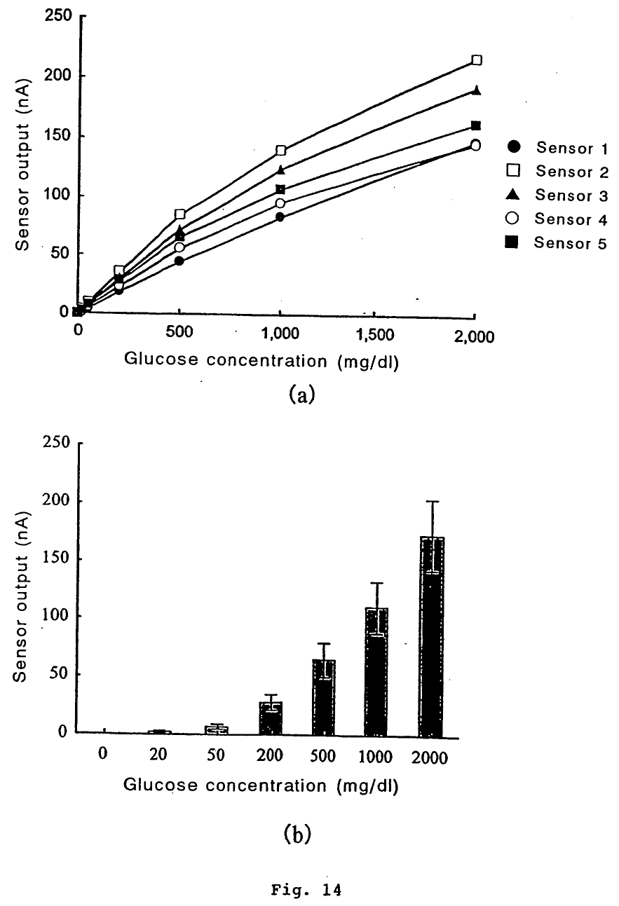

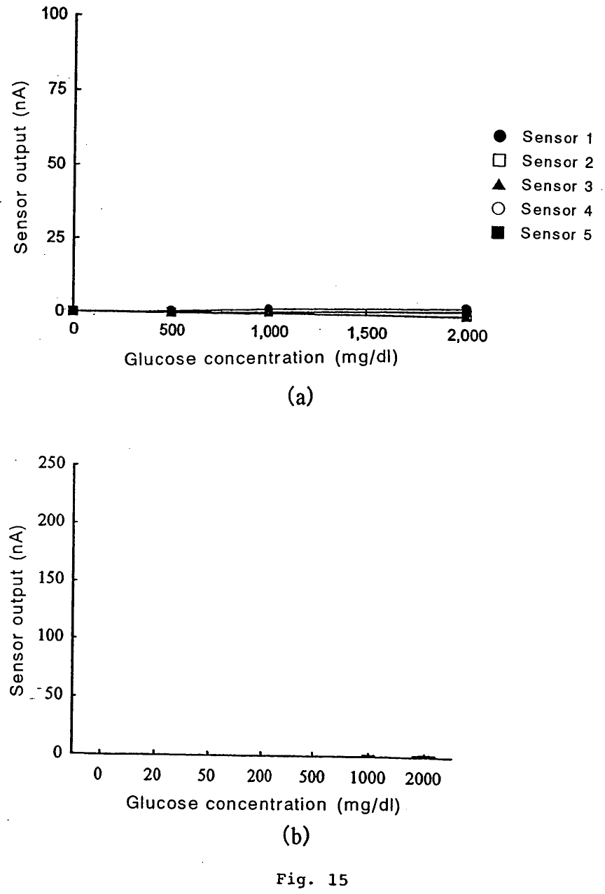

FIG. 12 shows the measurement results for the enzyme electrode (as a control) without an adhesion layer 8 between the immobilized enzyme layer 4 and the permeation-limiting layer 6, while FIGS. 13 to 15 show the measurement results for the enzyme electrode with an adhesion layer 8 between the immobilized enzyme layer 4 and the permeation-limiting layer 6. FIGS. 13, 14 and 15 show the results obtained for the enzyme electrodes where the adhesion layer 8 was formed using three aqueous solutions of γ-aminopropyltriethoxysilane at 0.1 v/v %, 0.05 v/v % and 0.2 v/v %, respectively. In these FIGs, (a) shows a calibration curve for five enzyme electrodes for each type and (b) is a bar chart of an average in which an error bar is a standard deviation. Comparing these results, it has been found that the adhesion layer 8 formed between the immobilized enzyme layer 4 and the permeation-limiting layer 6 allows for preparing an enzyme electrode with a smaller variation among the enzyme electrodes, i. e., an enzyme electrode exhibiting uniform properties and giving a highly linear calibration curve. Particularly, it has been found that the optimal properties were achieved in the enzyme electrode where the adhesion layer 8 was formed using the 0.1 v/v % aqueous solution of γ-aminopropyltriethoxysilane shown in FIG. 13, in the light of a measurement sensitivity for each enzyme electrode, variation in a measured value and linearity.

### Example 5

As shown in FIG. 3, on a 4-inch quartz wafer 12 (thickness: 0.515 mm; Nippon Electric Glass Co., Ltd.) were formed 82 sets of electrode chip, each set of which had the configuration shown in FIG. 4 and comprised a working electrode 9 (area: 5 mm²), a counter electrode 10 (area: 5 mm²) made of platinum and a reference electrode 11 (area: 1 mm²) made of silver/silver chloride. When cutting into the individual sets, the size of each electrode chip is 10 mm×6 mm. Then, the chip was immersed in a 6M solution of urea containing 150 mM sodium chloride, and 0.7 V was applied to the working electrode 9 in relation to the reference electrode 11 for 10 min. In practice, all the working electrodes 9 were interconnected as shown in FIG. 3 and connected to the periphery. Thus, the periphery and the reference electrode 11 were connected to an electrochemical measuring apparatus, and the above potential was applied. Thus, an urea layer as an electrode protective layer 2 was formed on the working electrode 9 by electrolysis.

Then, a 1 v/v % aqueous solution of γ-aminopropyltriethoxysilane was spin-coated and dried at 40° C. for 1 hour under nitrogen atmosphere to form a binding layer 3. Then, a 5 w/v % solution of Nafion was spin-coated and dried at 40° C. for 1 hour under nitrogen atmosphere to form an ion-exchange resin layer 7 mainly made of Nafion on the binding layer 3. Then, a 22.5 w/v % solution of albumin containing glucose oxidase and 1 v/v % glutaraldehyde was spin-coated to form an immobilized enzyme layer 4.

Then, as a silane coupling agent solution used in forming an adhesion layer 8 by spin coating, seven solutions were prepared by dissolving 0.1 v/v % of one of the following coupling agents (a) to (g) in a mixed solvent of the final concentration of 5 v/v % ethanol in pure water:

(a) γ-aminopropyltriethoxysilane

(b) γ-aminopropyltrimethoxysilane

(c) N-phenyl-γ-aminopropyltrimethoxysilane

(d) γ-chloropropyltrimethoxysilane

(e) γ-mercaptopropyltrimethoxysilane

(f) 3-isocyanatopropyltriethoxysilane

(g) 3-acryloxypropyltrimethoxysilane.

One of the seven solutions was spin-coated on each wafer and dried at 40° C. for 1 hour under nitrogen atmosphere to form adhesion layers 8 made of different silane coupling agents.

Subsequently, a 0.3 wt % 1H,1H-perfluorooctyl polymethacrylate solution prepared using perfluorohexane as a solvent was spin-coated on each of the seven wafers to form a permeation-limiting layer 6 made of 1H,1H-perfluorooctyl polymethacrylate. Thus enzyme electrode wafers were prepared.

Finally, the wafer was diced with a glass scribing apparatus to provide enzyme electrodes. Five of the enzyme electrode chips prepared were appropriately selected. Each chip was connected to a flexible substrate via a wire bonding and then the connecting part was waterproofed.

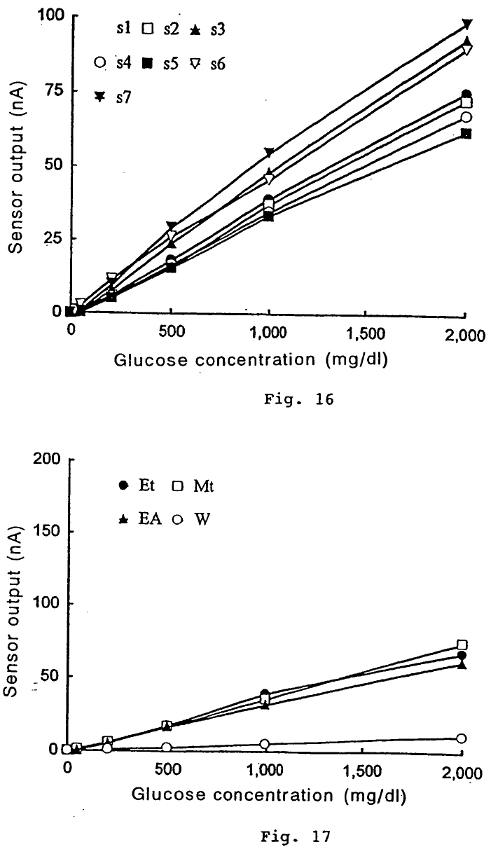

The seven types of enzyme electrodes thus prepared were stored by immersing them in a pH7 buffer of TES (N-tris(hydroxymethyl)methyl-2-aminoethanesulfonic acid) containing 150 mM sodium chloride. For a solution of glucose at 0 to 2000 mg/dl containing the TES buffer, a current as a sensor output was measured and a calibration curve was plotted for each of the five enzyme electrodes. Furthermore, for each of the seven types of enzyme electrodes, an average in the calibration curve for the five enzyme electrodes was calculated. FIG. 16 shows the results obtained by plotting the averages to a glucose concentration as an average calibration curve. In FIG. 16, s1 to s7 indicate enzyme electrodes having adhesion layers 8 prepared from the following coupling agents;

s1: (a) γ-aminopropyltriethoxysilane,

s2: (b) γ-aminopropyltrimethoxysilane,

s3: (c) N-phenyl-γ-aminopropyltrimethoxysilane,

s4: (d) γ-chloropropyltrimethoxysilane,

s5: (e) γ-mercaptopropyltrimethoxysilane,

s6: (f) 3-isocyanatopropyltriethoxysilane, and

s7: (g) 3-acryloxypropyltrimethoxysilane. Although a current value and linearity in a calibration curve vary to some extent depending on the type of a coupling agent used for preparing an adhesion layer 8, any of the coupling agents (a) to (g) may be used to achieve an adequate current value to a low level of glucose in an enzyme electrode prepared. That is, it has been found that an enzyme electrode by which a low level of glucose can be precisely measured can be prepared.

### Example 6

As shown in FIG. 3, on a 4-inch quartz wafer 12 (thickness: 0.515 mm; Nippon Electric Glass Co., Ltd.) were formed 82 sets of electrode chip, each set of which had the configuration shown in FIG. 4 and comprised a working electrode 9 (area: 5 mm²), a counter electrode 10 (area: 5 mm²) made of platinum and a reference electrode 11 (area: 1 mm²) made of silver/silver chloride. When cutting into the individual sets, the size of each electrode chip is 10 mm×6 mm. Then, the chip was immersed in a 6M solution of urea containing 150 mM sodium chloride, and 0.7 V was applied to the working electrode 9 in relation to the reference electrode 11 for 10 min. In practice, all the working electrodes 9 were interconnected as shown in FIG. 3 and connected to the periphery. Thus, the periphery and the reference electrode 11 were connected to an electrochemical measuring apparatus, and the above potential was applied. Thus, a urea layer as an electrode protective layer 2 was formed on the working electrode 9 by electrolysis.

Then, a 1 v/v % aqueous solution of γ-aminopropyltriethoxysilane was spin-coated and dried at 40° C. for 1 hour under nitrogen atmosphere to form a binding layer 3. Then, a 5 w/v % solution of Nafion was spin-coated and dried at 40° C. for 1 hour under nitrogen atmosphere to form an ion-exchange resin layer 7 mainly made of Nafion on the binding layer 3. Then, a 22.5 w/v % solution of albumin containing glucose oxidase and 1 v/v % glutaraldehyde was spin-coated to form an immobilized enzyme layer 4.

Then, as a silane coupling agent solution used in forming an adhesion layer 8 by spin coating, three solutions were prepared by dissolving 0.1 v/v % of γ-aminopropyltriethoxysilane in three different mixed solvents of the final concentration of 5 v/v % ethanol, methanol and ethyl acetate in pure water. Furthermore, as a control, a 0.1 v/v % solution of γ-aminopropyltiethoxysilane in pure water. One of the four solutions was spin-coated on each wafer and dried at 40° C. for 1 hour under nitrogen atmosphere to form adhesion layers 8.

Subsequently, a 0.3 wt % 1H,1H-perfluorooctyl polymethacrylate solution prepared using perfluorohexane as a solvent was spin-coated on each of the four wafers to form a permeation-limiting layer 6 made of 1H,1H-perfluorooctyl polymethacrylate. Thus enzyme electrode wafers were prepared.

Finally, the wafer was diced with a glass scribing apparatus to provide enzyme electrodes. Five of the enzyme electrode chips prepared were appropriately selected. Each chip was connected to a flexible substrate via a wire bonding and then the connecting part was waterproofed.

The four types of enzyme electrodes thus prepared were stored by immersing them in a pH7 buffer of TES (N-tris(hydroxymethyl)methyl-2-aminoethanesulfonic acid) containing 150 mM sodium chloride. For a solution of glucose at 0 to 2000 mg/dl containing the TES buffer, a current as a sensor output was measured and a calibration curve was plotted for each of the five enzyme electrodes. Furthermore, for each of the four types of enzyme electrodes, an average in the calibration curve for the five enzyme electrodes was calculated. FIG. 17 shows the results obtained by plotting the averages to a glucose concentration as an average calibration curve. In FIG. 17, the four symbols of Et, Mt, EA, and W indicate enzyme electrodes having adhesion layers 8 prepared from the γ-aminopropyltriethoxysilane solutions containing Et: ethanol in pure water, Mt: methanol in pure water, EA: ethyl acetate in pure water and W: pure water as a solvent, respectively. Difference in a current value and linearity in a calibration curve depending on the type of a mixed solvent are small, and in comparison with an electrode using pure water as a solvent, any of the mixed solvents prepared by adding one of the organic solvents to pure water in a small amount gave a significantly higher current value even for a low glucose concentration. That is, the step of forming the adhesion layer 8 made of a silane coupling agent can be conducted employing a mixed solvent in which an organic solvent is added to pure water, with a similar effect to that obtained employing a mixed solvent in which ethanol is added to pure water in a small amount as described in Example 5. Furthermore, it has been found that by using a solvent containing any organic solvent in pure water within in a concentration range equivalent to a final concentration of 5 v/v % described in Example 6, for example 7 v/v % or less of the final concentration, an enzyme electrode by which a low level of glucose can be precisely measured can be manufactured.

### Example 7

As shown in FIG. 3, on a 4-inch quartz wafer 12 (thickness: 0.515 mm; Nippon Electric Glass Co., Ltd.) were formed 82 sets of electrode chip, each set of which had the configuration shown in FIG. 4 and comprised a working electrode 9 (area: 5 mm²), a counter electrode 10 (area: 5 mm²) made of platinum and a reference electrode 11 (area: 1 mm²) made of silver/silver chloride. When cutting into the individual sets, the size of each electrode chip is 10 mm×6 mm. Then, the chip was immersed in a 6M solution of urea containing 150 mM sodium chloride, and 0.7 V was applied to the working electrode 9 in relation to the reference electrode 11 for 10 min. In practice, all the working electrodes 9 were interconnected as shown in FIG. 3 and connected to the periphery. Thus, the periphery and the reference electrode 11 were connected to an electrochemical measuring apparatus, and the above potential was applied. Thus, an urea layer as an electrode protective layer 2 was formed on the working electrode 9 by electrolysis.

Then, a 1 v/v % aqueous solution of γ-aminopropyltriethoxysilane was spin-coated and dried at 40° C. for 1 hour under nitrogen atmosphere to form a binding layer 3. Then, a 5 w/v % solution of Nafion was spin-coated and dried at 40° C. for 1 hour under nitrogen atmosphere to form an ion-exchange resin layer 7 mainly made of Nafion on the binding layer 3. Then, a 22.5 w/v % solution of albumin containing glucose oxidase and 1 v/v % glutaraldehyde was spin-coated to form an immobilized enzyme layer 4.

Then, on the wafer was spin-coated a 0.1 v/v % solution of γ-aminopropyltriethoxysilane prepared in a mixed solvent in which ethanol was added to pure water to the final concentration of 5 v/v %, and the wafer was dried at 40° C. for 1 hour under nitrogen atmosphere to form an adhesion layer 8.

Subsequently, a 0.3 wt % 1H,1H-perfluorooctyl polymethacrylate solution prepared using perfluorohexane as a solvent was spin-coated to form a permeation-limiting layer 6 made of 1H,1H-perfluorooctyl polymethacrylate. Thus enzyme electrode wafers were prepared.

Finally, the wafer was diced with a glass scribing apparatus to provide enzyme electrodes. Each of the enzyme electrode chips prepared was connected to a flexible substrate via a wire bonding and then the connecting part was waterproofed.

The enzyme electrodes thus prepared were stored by immersing them in a pH7 buffer of TES (N-tris(hydroxymethyl)methyl-2-aminoethanesulfonic acid) containing 150 mM sodium chloride. For a solution of glucose at 0 to 2000 mg/dl containing the TES buffer, a current as a sensor output was measured and a calibration curve was plotted for an enzyme electrode prepared from each of the chips formed on the wafer as a matrix.

Additionally, for comparison, an enzyme electrode wafer without only adhesion layer 8 was formed as described above except that the step of forming the adhesion layer 8 was omitted, and enzyme electrodes without an adhesion layer 8 were prepared. For these electrodes, a calibration curve was plotted for an enzyme electrode prepared from each chip formed on the wafer as a matrix.

For the individual enzyme electrodes, acceptable enzyme electrodes were selected on the basis of the calibration curve according to the following criteria. An acceptable enzyme electrode meets both of the following selection criteria that it has an output of 30 nA or more to less than 150 nA to a glucose concentration of 2000 mg/dl for sensitivity and that an output to a glucose concentration of 500 m g/dl is within ¼±30% to an output to a glucose concentration of 2000 mg/dl for linearity of a calibration curve. Among 82 enzyme electrodes formed from each chip on each wafer, acceptable enzyme electrodes were selected and a yield was calculated from the calculation equation below.

Calculation equation: Yield (%)=Acceptable products/Total×100

For the purpose of the measurement results of an output to a glucose concentration of 2000 mg/dl obtained using the selection criteria, Table 2 shows a sensor output from an enzyme electrode without an adhesion layer while Table 3 shows a sensor output from an enzyme electrode with an adhesion layer. In these tables, an in-plane position of each chip formed as a matrix on a wafer is indicated by a combination of an alphabet and a number. For example, an enzyme electrode formed from the chip at a position indicated by a combination of “A” and “3” in Table 2 has a sensor output of “43.6”. From the evaluation results, yields were about 32% (26/82) and about 85% (70/82) for the enzyme electrodes without and with an adhesion layer, respectively. The comparison results described above demonstrate that in a mass production process using a wafer process, employing an enzyme electrode structure comprising an adhesion layer is effective for improving a yield of acceptable products per a wafer.

TABLE 2
Outputs for a glucose concentration of 2000 mg/dl: in -plane
distribution for a wafer (nA)

| | A | B | C | D | E | F | G | H | I | J | K |
|---|---|---|---|---|---|---|---|---|---|---|---|
| 1 | 19.5 | 10.0 | 4.0 | 69.3 | 20.5 | 35.5 | 20.8 | 4.3 | 52.7 | — | — |
| 2 | 29.5 | 26.4 | 10.3 | 64.5 | 15.4 | 56.3 | 11.2 | 10.1 | 34.0 | 13.0 | — |
| 3 | 43.6 | 43.6 | 9.4 | 17.2 | 49.5 | 37.2 | 4.8 | 3.0 | 16.3 | 19.8 | 17.0 |
| 4 | 69.7 | 5.0 | 59.4 | 15.1 | 13.6 | 13.0 | 25.8 | 12.1 | 5.6 | 32.0 | 4.2 |
| 5 | 44.7 | 44.7 | 26.0 | 25.2 | 17.3 | 35.5 | 18.7 | 4.2 | 5.5 | 9.8 | 32.9 |
| 6 | 65.6 | 10.0 | 37.1 | 9.2 | 19.1 | 42.9 | 34.6 | 9.8 | 4.2 | 9.9 | 56.3 |
| 7 | 99.3 | 25.7 | 9.5 | 16.9 | 13.0 | 19.4 | 20.0 | 9.7 | 11.2 | 25.1 | — |
| 8 | 8.0 | 10.0 | 6.0 | 4.0 | 60.4 | 33.4 | 13.6 | 9.9 | 30.7 | — | — |

TABLE 3
Outputs for a glucose concentration of 2000 mg/dl: in -plane
distribution for a wafer (nA)

| | A | B | C | D | E | F | G | H | I | J | K |
|---|---|---|---|---|---|---|---|---|---|---|---|
| 1 | 44.0 | 46.6 | 46.7 | 70.2 | 44.1 | 33.3 | 20.1 | 39.1 | 50.0 | — | — |
| 2 | 30.1 | 26.1 | 46.1 | 65.1 | 12.9 | 59.3 | 10.0 | 11.9 | 44.1 | 33.4 | — |
| 3 | 44.1 | 46.0 | 43.1 | 99.9 | 49.8 | 39.2 | 77.1 | 99.0 | 32.0 | 44.1 | 32.0 |
| 4 | 72.0 | 99.9 | 65.4 | 39.7 | 11.1 | 11.9 | 53.0 | 43.0 | 60.4 | 32.0 | 52.1 |
| 5 | 47.0 | 45.9 | 25.7 | 77.7 | 43.9 | 45.6 | 44.4 | 35.1 | 64.5 | 52.4 | 32.9 |
| 6 | 69.2 | 64.0 | 40.1 | 37.1 | 18.4 | 44.9 | 40.0 | 99.0 | 69.7 | 9.9 | 56.3 |
| 7 | 99.7 | 98.9 | 44.1 | 15.4 | 38.7 | 18.9 | 46.0 | 43.0 | 33.4 | 69.7 | — |
| 8 | 39.4 | 70.0 | 39.4 | 43.3 | 62.7 | 35.4 | 34.4 | 52.4 | 34.6 | — | — |

### Example 8

As shown in FIG. 3, on a 4-inch quartz wafer 12 (thickness: 0.515 mm; Nippon Electric Glass Co., Ltd.) were formed 82 sets of electrode chip, each set of which had the configuration shown in FIG. 4 and comprised a working electrode 9 (area: 5 mm²), a counter electrode 10 (area: 5 mm²) made of platinum and a reference electrode 11 (area: 1 mm²) made of silver/silver chloride. When cutting into the individual sets, the size of each electrode chip is 10 mm×6 mm. Then, the chip was immersed in a 6M solution of urea containing 150 mM sodium chloride, and 0.7 V was applied to the working electrode 9 in relation to the reference electrode 11 for 10 min. In practice, all the working electrodes 9 were interconnected as shown in FIG. 3 and connected to the periphery. Thus, the periphery and the reference electrode 11 were connected to an electrochemical measuring apparatus, and the above potential was applied. Thus, an urea layer as an electrode protective layer 2 was formed on the working electrode 9 by electrolysis.

Then, a 1 v/v % aqueous solution of γ-aminopropyltriethoxysilane was spin-coated to form a binding layer 3. Then, a 5 w/v % solution of a perfluorocarbonsulfonic acid resin was spin-coated to form an ion-exchange resin layer 7 mainly made of the perfluorocarbonsulfonic acid resin (Nafion) on the binding layer 3. Then, a 22.5 w/v % solution of albumin containing glucose oxidase and 1 v/v % glutaraldehyde was spin-coated to form an immobilized enzyme layer 4. Then, a 0.1 v/v % aqueous solution of γ-aminopropyltriethoxysilane was spin-coated to form an adhesion layer 8. Subsequently, a 0.1, 0.3, 1.0 or 10 wt % 1H,1H-perfluorooctyl polymethacrylate solution prepared using perfluorohexane as a solvent was spin-coated to form a permeation-limiting layer 6 made of 1H,1H-perfluorooctyl polymethacrylate with a corresponding thickness, respectively. Thus four types of enzyme electrode wafers were prepared.

The spin-coating conditions were as follows; a rotation speed of 3000 rpm and a rotation time of 30 sec under 4° C. atmosphere.

Finally, the wafer was diced with a glass scribing apparatus to provide enzyme electrodes, from which five chips were then appropriately selected for each type. Each of the enzyme electrode chips prepared was connected to a flexible substrate via a wire bonding and then the connecting part was waterproofed.

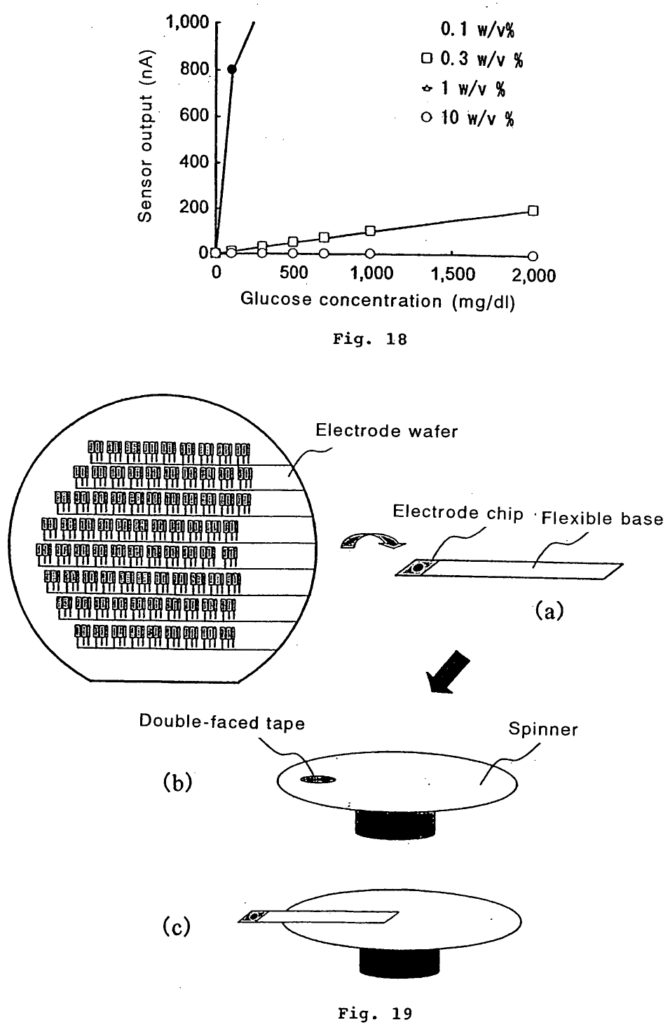

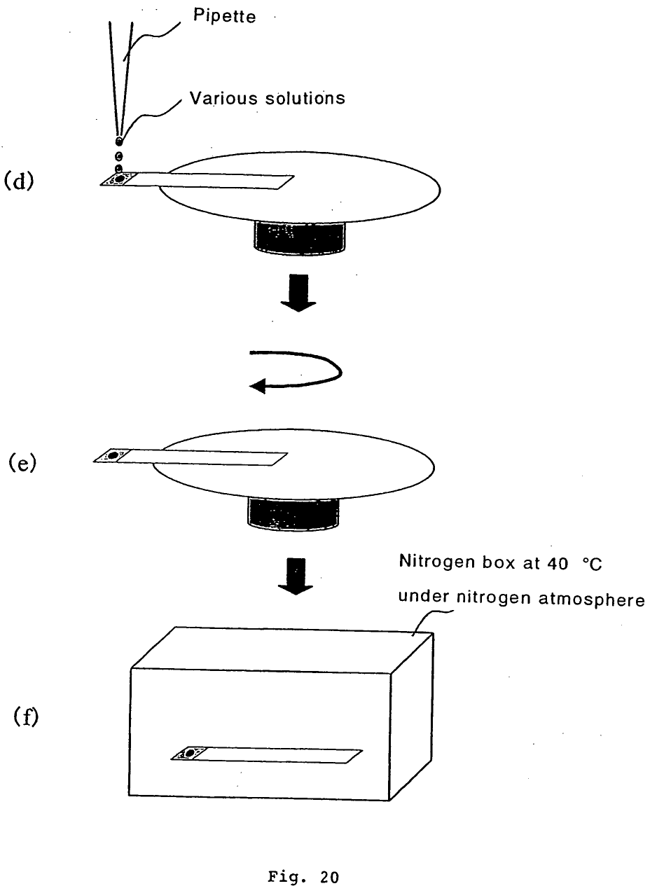

The enzyme electrodes thus prepared were stored by immersing them in a pH7 buffer of TES (N-tris(hydroxymethyl)methyl-2-aminoethanesulfonic acid) containing 150 mM sodium chloride, and current values for 0 to 2000 mg/dl glucose solutions were measured. FIG. 18 shows the measured values as an average of five electrodes.

The above solution of 1H,1H-perfluorooctyl polymethacrylate at the above four concentrations was spin-coated on a quartz wafer surface with the same size as described above to form samples for evaluating a film thickness of a permeation-limiting layer. Then, the wafers were cut into pieces with the same size as that of the enzyme electrode chip by dicing the wafers using a glass scribing apparatus. Then, a part of a permeation-limiting layer on the evaluation sample was peeled using an ultrasonic cutter to expose the quartz glass surface. Then, using an atomic force microscope (SPI 3000 from Seiko Instrument), a step between the quartz glass surface and the surface of the permeation-limiting layer (measurement at n=5) to determine a thickness of the permeation-limiting layer. Table 4 shows a measured thickness for the permeation-limiting layer.

TABLE 4
Thickness of the permeation-limiting layer

| Solution | Thickness of the |
|---|---|
| concentration | permeation-limiting |
| (wt %) | layer (nm) |
| 0.1 | 10 |
| 0.3 | 70 |
| 1.0 | 250 |
| 10.0 | 600 |

From comparison of the results shown in FIG. 18 with the measurement results of a film thickness summarized in Table 4, it may be supposed that using a solution of 1H,1H-perfluorooctyl polymethacrylate at 1 wt % or more, the permeation-limiting layer formed had a thickness of 250 nm or more so that permeation-limiting effect was abruptly improved, resulting in an extremely lower current as a sensor output. However, since output linearity was retained, the permeation-limiting layer seemed to be uniformly formed.

Thus, in the light of output linearity, it is indicated that a thickness of a permeation-limiting layer exhibiting suitable permeation control is 70 nm.

Examples 9 to 12 presented below represent the most preferred embodiments in the second aspect of the present invention, but the second aspect of the present invention is not limited to these specific examples.

### Example 9

Two pieces of 4-inch wafers (Nippon Electric Glass Co., Ltd.) with a thickness of 0.515 mm were prepared and used for the following procedure.

As shown in FIG. 3, on the quartz wafer 12 were formed 87 sets of electrode chip, each set of which had the configuration shown in FIG. 4 and comprised a working electrode 9 (area: 5 mm²), a counter electrode 10 (area: 5 mm²) made of platinum and a reference electrode 11 (area: 1 mm²) made of silver/silver chloride. When cutting into the individual sets, the size of each electrode chip is 10 mm×6 mm. All the working electrodes 9 were interconnected as shown in FIG. 3 and connected to the periphery.

Then, a 1 v/v % aqueous solution of γ-aminopropyltriethoxysilane (hereinafter, referred to as “APTES”) was spin-coated to form a binding layer 3. Then, a 22.5 w/v % solution of albumin containing glucose oxidase and 1 v/v % glutaraldehyde was spin-coated to form an immobilized enzyme layer 4.

Then, on the immobilized enzyme layer 4 was applied a 0.3 wt % 1H,1H-perfluorooctyl polymethacrylate solution prepared using perfluorohexane as a solvent and the applied film was dried to form a permeation-limiting layer 6 made of 1H,1H-perfluorooctyl polymethacrylate.

For one wafer, the applied film was formed by spin coating under the following conditions. A sample prepared by the spin-coating process is referred to as “Sample 1”.

Spin-coating rotation speed: 3000 rpm for 30 sec;

Solution volume added: 0.3 μl/mm²;

Deposition temperature (solution temperature): 4° C.

For the other wafer, the applied film was formed by dip-coating. The sample prepared by this dip-coating is referred to as “Sample 2”.

Finally, the wafer was diced with a glass scribing apparatus to provide enzyme electrodes. For the two enzyme electrode chips prepared by the different application methods of a polymer material constituting the permeation-limiting layer 6, a film thickness and a surface roughness of the permeation-limiting layer 6 were determined and the electrode surface coated with the permeation-limiting layer 6 was observed by an atomic force microscope to determine its surface roughness. The results are as follows.

Sample 1 (Spin Coating)

Average film thickness D: 0.3 μm; surface roughness R: 0.6 nm; R/D=0.002.

Sample 2 (Dip Coating)

Average film thickness D: 1.4 μm; surface roughness R: 1.3 nm; R/D=0.0009.

A surface roughness is an median (R50).

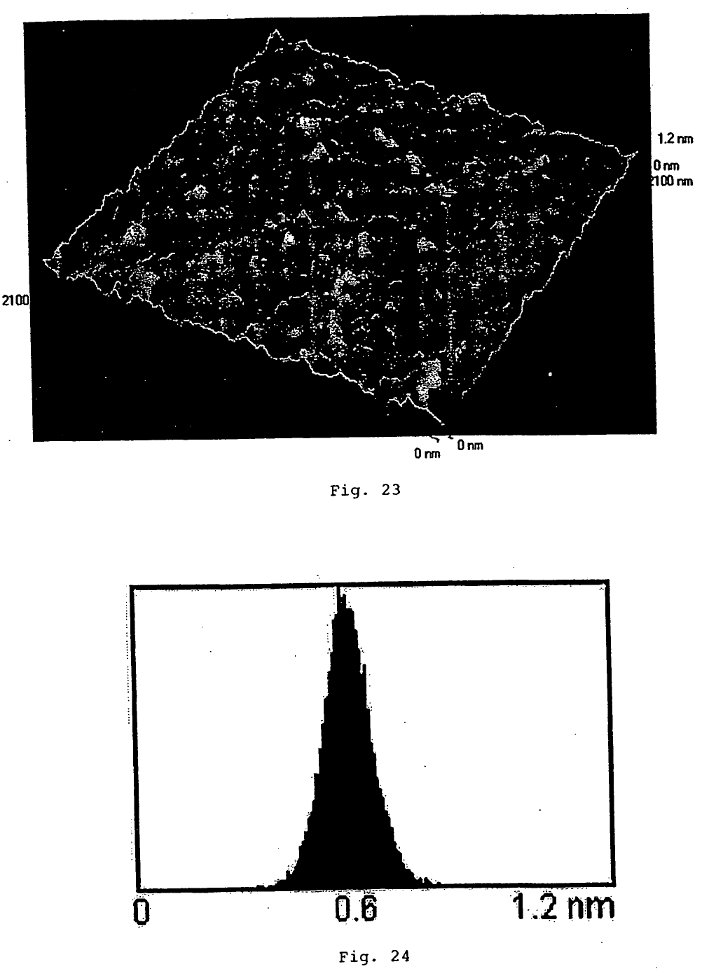

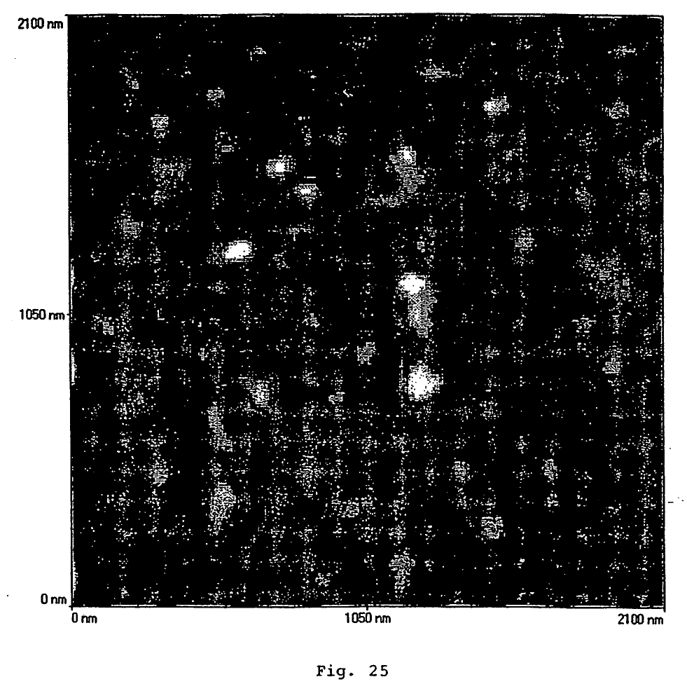

For Sample 1 formed by spin coating, the overall surface of the permeation-limiting layer 6 has fine grooves. FIG. 23 is a printout of surface AFM (AFM: Atomic Force Microscopy) image for the grooves formed in the surface of the permeation-limiting layer 6. FIG. 24 shows a surface roughness in a corresponding permeation-limiting layer, which is determined based on the surface AFM image shown in FIG. 23. Groove depths exhibit a distribution centering the roughness, and all of these are within the range of 0.1 to 100 nm.

FIG. 25 shows an example of a printout for an observed image by AFM for the permeation-limiting layer. In this figure, a white part indicates a groove formed on the permeation-limiting layer. It has been observed that the surface of the permeation-limiting layer 6 formed by the above manufacturing process are randomly has a number of grooves.

### Example 10

Two pieces of 4-inch wafers (Nippon Electric Glass Co., Ltd.) with a thickness of 0.515 mm were prepared and used for the following procedure.

As shown in FIG. 3, on the quartz wafer 12 were formed 87 sets of electrode chip, each set of which had the configuration shown in FIG. 4 and comprised a working electrode 9 (area: 5 mm²), a counter electrode 10 (area: 5 mm²) made of platinum and a reference electrode 11 (area: 1 mm²) made of silver/silver chloride. When cutting into the individual sets, the size of each electrode chip is 10 mm×6 mm. All the working electrodes 9 were interconnected as shown in FIG. 3 and connected to the periphery.

Then, a 1 v/v % aqueous solution of γ-aminopropyltriethoxysilane was spin-coated to form a binding layer 3. Then, a 22.5 w/v % solution of albumin containing glucose oxidase and 1 v/v % glutaraldehyde was spin-coated to form an immobilized enzyme layer 4.

Then, for one wafer, on the immobilized enzyme layer 4 was applied by spin coating a 0.3 wt % solution of a fluoroalcohol ester of an acrylic resin prepared using xylene hexafluoride as a solvent and the applied film was dried to form a permeation-limiting layer 6 made of the fluoroalcohol ester of the acrylic resin. The conditions of spin coating were a rotation speed: 3000 rpm and a time: 30 sec. The applied solution was prepared by further adding a solvent, xylene hexafluoride, to a solution of 1H,1H,2H,2H-perfluorodecyl polyacrylic acid in xylene hexafluoride (the acrylic resin: 17%, xylene hexafluoride: 83%, viscosity: 20 cps at 25° C.) to give a diluted solution with a resin content of 0.3 wt %. Finally, the wafer was diced with a glass scribing apparatus to prepare the first enzyme electrode.

For the other wafer as a control, on the immobilized enzyme layer 4 was applied by dip coating a solution of a fluoroalcohol ester of an acrylic resin and then drying the applied film to form a permeation-limiting layer 6 made of the fluoroalcohol ester of the acrylic resin. As described above, the wafer was diced with a glass scribing apparatus to prepare the second enzyme electrode.

For each of the first and the second enzyme electrodes, four chips were randomly taken from the wafer for the following evaluation. An average thickness of the permeation-limiting layer 6 was 0.08 μm for the first enzyme electrode while being 1.6 μm for the second enzyme electrode. For the first enzyme electrode, the profile of the permeation-limiting layer 6 determined based on a surface AFM image was as follows.

Average film thickness D: 0.08 μm, Surface roughness R: 0.6 nm, R/D=0.0075.

Each enzyme electrode chip was connected to an electrochemical measurement apparatus by wire bonding and immersed at 24° C. in a pH-adjusted solution using a pH7 TES (N-tris(hydroxymethyl)methyl-2-aminoethanesulfonic acid) buffer containing 150 mM sodium chloride. Then, a difference between a base current when a voltage was applied to a solution without a target compound and an output current for the solution without the target compound was measured as a sensor output. The applied voltage was 700 mV at a working electrode in relation to a reference electrode. Not only during measurement but also during storage, each enzyme electrode was immersed in the pH7 TES buffer containing 150 mM sodium chloride. Sensor output values for solutions containing the TES buffer at a glucose concentration of 0 to 2000 mg/dl were measured and a calibration curve was plotted for four enzyme electrodes of one type.

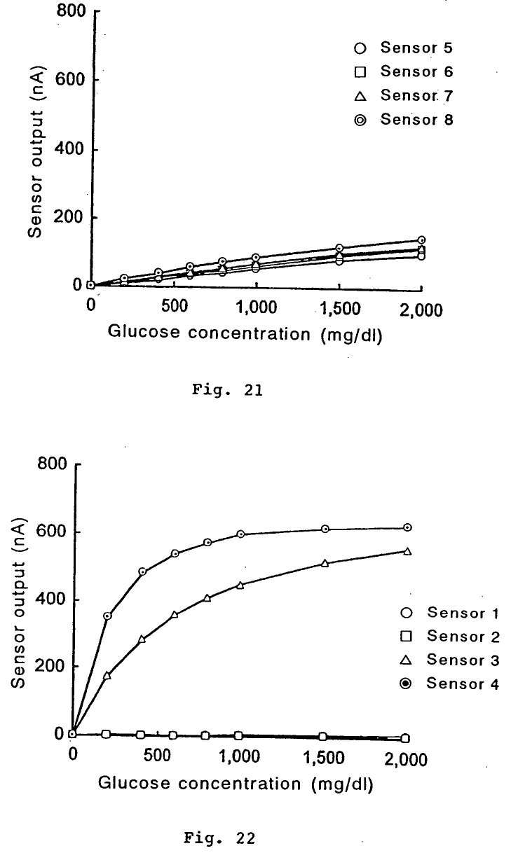

FIG. 21 shows a sensor output (calibration curve) to glucose in the first enzyme electrode (spin coating). FIG. 22 shows a sensor output (calibration curve) to glucose in the second enzyme electrode (dip coating). For the first enzyme electrode where the permeation-limiting layer 6 was formed by spin coating, a highly linear sensor output was obtained and little variation in an output between sensors was observed. The improved selective permeability may be achieved by forming the permeation-limiting layer 6 by spin coating to make grooves on the surface, which allows glucose to smoothly permeate to the immobilized enzyme layer 4. In contrast, when the permeation-limiting layer 6 was formed by dip coating, linearity in a sensor output (calibration curve) was reduced and variation in an output between sensors was increased. It may be because no grooves are formed in the surface of the permeation-limiting layer 6 using dip coating so that glucose permeation may be not facilitated, permeability variation may be increased and performance variation in each enzyme electrode chip formed in the wafer plane may be increased.

From the above comparison, when forming the permeation-limiting layer 6 by spin coating to provide an enzyme electrode comprising the permeation-limiting layer 6 having grooves in its surface, it has been demonstrated that an enzyme electrode formed exhibits excellent properties such as a highly linear calibration curve and smaller variation between sensors.

### Example 11

Two pieces of 4-inch wafers (Nippon Electric Glass Co., Ltd.) with a thickness of 0.515 mm were prepared and used for the following procedure.

As shown in FIG. 3, on the quartz wafer 12 were formed 87 sets of electrode chip, each set of which had the configuration shown in FIG. 4 and comprised a working electrode 9 (area: 5 mm²), a counter electrode 10 (area: 5 mm²) made of platinum and a reference electrode 11 (area: 1 mm²) made of silver/silver chloride. When cutting into the individual sets, the size of each electrode chip is 10 mm×6 mm. All the working electrodes 9 were interconnected as shown in FIG. 3 and connected to the periphery.

Then, a 1 v/v % aqueous solution of γ-aminopropyltriethoxysilane was spin-coated to form a binding layer 3. Then, a 22.5 w/v % solution of albumin containing glucose oxidase and 1 v/v % glutaraldehyde was spin-coated to form an immobilized enzyme layer 4. On the immobilized enzyme layer 4 was spin-coated a 1 v/v % aqueous solution of γ-aminopropyltriethoxysilane to form an adhesion layer 8.

Then, for one wafer, on the immobilized enzyme layer 4 on which the adhesion layer 8 was formed was applied by spin coating a 0.3 wt % solution of a fluoroalcohol ester of an acrylic resin prepared using xylene hexafluoride as a solvent and the applied film was dried to form a permeation-limiting layer 6 made of the fluoroalcohol ester of the acrylic resin. The conditions of spin coating were a rotation speed: 3000 rpm and a time: 30 sec. The applied solution was prepared by further adding a solvent, xylene hexafluoride, to a solution of 1H,1H,2H,2H-perfluorodecyl polyacrylic acid in xylene hexafluoride (the acrylic resin: 17%, xylene hexafluoride: 83%, viscosity: 20 cps at 25° C.) to give a diluted solution with a resin content of 0.3 wt %. Finally, the wafer was diced with a glass scribing apparatus to prepare the first enzyme electrode.

For the other wafer as a control, on the immobilized enzyme layer 4 was applied by dip coating a solution of a fluoroalcohol ester of an acrylic resin and then drying the applied film to form a permeation-limiting layer 6 made of the fluoroalcohol ester of the acrylic resin. As described above, the wafer was diced with a glass scribing apparatus to prepare the second enzyme electrode.

For each of the first and the second enzyme electrodes, three chips were randomly taken from the wafer for the following evaluation. An average thickness of the permeation-limiting layer 6 was 0.2 μm for the first enzyme electrode while being 1.4 μm for the second enzyme electrode. For the first enzyme electrode, the profile of the permeation-limiting layer 6 determined based on a surface AFM image was as follows.

Average film thickness D: 0.2 μm, Surface roughness R: 0.5 nm, R/D=0.0025.

Each enzyme electrode chip was connected to an electrochemical measurement apparatus by wire bonding and immersed at 24° C. in a pH-adjusted solution using a pH7 TES (N-tris(hydroxymethyl)methyl-2-aminoethanesulfonic acid) buffer containing 150 mM sodium chloride. Then, a difference between a base current when a voltage was applied to a solution without a target compound and an output current for the solution without the target compound was measured as a sensor output. The applied voltage was 700 mV at a working electrode in relation to a reference electrode. Not only during measurement but also during storage, each enzyme electrode was immersed in the pH7 TES buffer containing 150 mM sodium chloride. For example, sensor output values for solutions containing the TES buffer at a glucose concentration of 0 to 2000 mg/dl were measured and a calibration curve to glucose was plotted for four enzyme electrodes of one type.

For the first enzyme electrode (spin coating), using the three sensors for which a calibration curve was plotted, components in real urine from diabetics (22 samples) were measured. Separately, using an existing standard apparatus (trade name: Hitachi Automatic Measurement Apparatus 7050), components in the real urine from the diabetics (22 samples) were measured under the same conditions. The measured values from the first enzyme electrode was subject to regression analysis to the contents of the individual components in the real urine (22 samples) determined by measurement using the existing apparatus and a correlation coefficient to the measured value from the existing standard apparatus was calculated for evaluation. Similarly, the second enzyme electrode (dip coating) was used to determine components in the real urine from the diabetics, and after regression analysis, a correlation coefficient to the measured value from the existing apparatus was calculated for evaluation. Table 5 shows a correlation coefficient calculated from the evaluation results for each enzyme electrode.

For the first enzyme electrode where the permeation-limiting layer 6 was formed by spin coating, every electrode gave higher correlation with a R of 0.99 or more. On the other hand, for the second enzyme electrode where the permeation-limiting layer 6 was formed by dip coating, variation in a correlation coefficient between the electrodes was observed and a correlation coefficient R was 0.89 or less in any case.

When forming the permeation-limiting layer 6 by spin coating to provide an enzyme electrode comprising the permeation-limiting layer 6 where a number of grooves are formed in its surface, the enzyme electrode has uniform grooves and glucose smoothly permeates. Furthermore, the electrode is endowed appropriate surface roughness, so that adhesion of contaminants can be minimized and thus performance of the individual enzyme electrodes prepared in the wafer plane may be uniform.

From the above comparison, when forming the permeation-limiting layer 6 by spin coating to prepare an enzyme electrode comprising the permeation-limiting layer 6, it is demonstrated that such an enzyme electrode improved measurement precision equivalent to that in measurement using an existing large apparatus for laboratory testing.

TABLE 5
The first enzyme electrode

| | |
|---|---|
| Sensor 1-1 | R = 0.990 |
| Sensor 1-2 | R = 0.997 |
| Sensor 1-3 | R = 0.994 |

The second enzyme electrode

| | |
|---|---|
| Sensor 2-1 | R = 0.890 |
| Sensor 2-2 | R = 0.789 |
| Sensor 2-3 | R = 0.819 |

### Example 12

Two pieces of 4-inch wafers (Nippon Electric Glass Co., Ltd.) with a thickness of 0.515 mm were prepared and used for the following procedure.

As shown in FIG. 3, on the quartz wafer 12 were formed 87 sets of electrode chip, each set of which had the configuration shown in FIG. 4 and comprised a working electrode 9 (area: 5 mm²), a counter electrode 10 (area: 5 mm²) made of platinum and a reference electrode 11 (area: 1 mm²) made of silver/silver chloride. When cutting into t he individual sets, the size of each electrode chip is 10 mm×6 mm. All the working electrodes 9 were interconnected as shown in FIG. 3 and connected to the periphery.

Then, a 1 v/v % aqueous solution of γ-aminopropyltriethoxysilane was spin-coated to form a binding layer 3. Then, a 22.5 w/v % solution of albumin containing glucose oxidase and 1 v/v % glutaraldehyde was spin-coated to form an immobilized enzyme layer 4. On the immobilized enzyme layer 4 was spin-coated a 1 v/v % aqueous solution of γ-aminopropyltriethoxysilane to form an adhesion layer 8.

Then, for one wafer, on the immobilized enzyme layer 4 on which the adhesion layer 8 was formed was applied by spin coating a 0.3 wt % solution of a fluoroalcohol ester of an acrylic resin prepared using xylene hexafluoride as a solvent and the applied film was dried to form a permeation-limiting layer 6 made of the fluoroalcohol ester of the acrylic resin. The conditions of spin coating were a rotation speed: 3000 rpm and a time: 30 sec. The applied solution was prepared by further adding a solvent, xylene hexafluoride, to a solution of 1H,1H,2H,2H-perfluorodecyl polyacrylic acid in xylene hexafluoride (the acrylic resin: 17%, xylene hexafluoride: 83%, viscosity: 20 cps at 25° C.) to give a diluted solution with a resin content of 0.3 wt %. Finally, the wafer was diced with a glass scribing apparatus to prepare the first enzyme electrode.

For the other wafer as a control, on the immobilized enzyme layer 4 was applied by dip coating a solution of a fluoroalcohol ester of an acrylic resin and then drying the applied film to form a permeation-limiting layer 6 made of the fluoroalcohol ester of the acrylic resin. As described above, the wafer was diced with a glass scribing apparatus to prepare the second enzyme electrode.

For each of the first and the second enzyme electrodes, three chips were randomly taken from the wafer for the following evaluation. An average thickness of the permeation-limiting layer 6 was 0.2 μm for the first enzyme electrode while being 1.4 μm for the second enzyme electrode. For the first enzyme electrode, the profile of the permeation-limiting layer 6 determined based on a surface AFM image was as follows.

Average film thickness D: 0.2 μm, Surface roughness R: 0.5 nm, R/D=0.0025.

Each enzyme electrode chip was connected to an electrochemical measurement apparatus by wire bonding and immersed at 24° C. in a pH-adjusted solution using a pH7 TES (N-tris(hydroxymethyl)methyl-2-aminoethanesulfonic acid) buffer containing 150 mM sodium chloride. Then, a difference between a base current when a voltage was applied to a solution without a target compound and an output current for the solution without the target compound was measured as a sensor output. The applied voltage was 700 mV at a working electrode in relation to a reference electrode. Not only during measurement but also during storage, each enzyme electrode was immersed in the pH7 TES buffer containing 150 mM sodium chloride. Then, sensor output values for solutions containing the TES buffer at a glucose concentration of 0 to 2000 mg/dl were measured and a calibration curve to glucose was plotted for four enzyme electrodes of one type.

For the first enzyme electrode (spin coating), using the three sensors for which a calibration curve was plotted, components in plasma from diabetics (31 samples) were measured. Separately, using an existing standard apparatus (trade name: Hitachi Automatic Measurement Apparatus 7050), components in the plasma from the diabetics (31 samples) were measured under the same conditions. The measured values from the first enzyme electrode was subject to regression analysis to the contents of the individual components in the plasma (31 samples) determined by measurement using the existing standard apparatus and a correlation coefficient to the measured value from the existing apparatus was calculated for evaluation. Similarly, the second enzyme electrode (dip coating) was used to determine components in the plasma from the diabetics, and after regression analysis, a correlation coefficient to the measured value from the existing apparatus was calculated for evaluation. Table 6 shows a correlation coefficient calculated from the evaluation results for each enzyme electrode.

The first enzyme electrode (spin coating) was used to analyze plasma from diabetics (31 samples) while a laboratory testing apparatus (trade name: Hitachi Automatic Measurement Apparatus 7050) as an existing standard apparatus was used to analyze the samples under the same conditions. The values obtained for the individual components were subject to regression analysis and a correlation coefficient was calculated for evaluation. Furthermore, the second enzyme electrode (dip coating) was used to analyze the plasma from the diabetics. The results are shown in Table 6.

For the first enzyme electrode where the permeation-limiting layer 6 was formed by spin coating, every electrode gave higher correlation with an R of 0.99 or more. On the other hand, for the second enzyme electrode where the permeation-limiting layer 6 was formed by dip coating, variation in a correlation coefficient between the electrodes was observed and a correlation coefficient R was 0.92 or less in any case.

When forming the permeation-limiting layer 6 by spin coating to provide an enzyme electrode comprising the permeation-limiting layer 6 where a number of grooves are formed in its surface, the enzyme electrode has uniform grooves and glucose smoothly permeates. Furthermore, the electrode is endowed appropriate surface roughness, so that adhesion of contaminants can be minimized and thus performance of the individual enzyme electrodes prepared in the wafer plane may be uniform. In additions although no data are demonstrated, complete removal of contaminants adhering an electrode surface, particularly the outermost surface, i. e., the permeation-limiting layer surface during cleaning after measurement may also contribute improvement in measurement precision in analysis of a number of samples.

From the above comparison, when forming the permeation-limiting layer 6 by spin coating to prepare an enzyme electrode comprising the permeation-limiting layer 6, it is demonstrated that such an enzyme electrode improved measurement precision equivalent to that in measurement using an existing large apparatus for laboratory testing.

TABLE 6
The first enzyme electrode

| | |
|---|---|
| Sensor 9-1 | R = 0.992 |
| Sensor 9-2 | R = 0.995 |
| Sensor 9-3 | R = 0.990 |

The second enzyme electrode

| | |
|---|---|
| Sensor 10-1 | R = 0.901 |
| Sensor 10-2 | R = 0.922 |
| Sensor 19-3 | R = 0.897 |

### INDUSTRIAL APPLICABILITY

As explained above, in the present invention, as an enzyme electrode according to the first aspect of the present invention has such a constitution in which an adhesion layer 8 comprising a silane-containing compound is equipped over an immobilized enzyme layer 4 and in contact with the upper surface of the adhesion layer 8, a permeation-limiting layer 6 comprising a fluorine-containing polymer having a particular structure is formed; and thereby the adhesion layer 8 comprising the silane-containing compound is lied between the immobilized enzyme layer 4 and the permeation-limiting layer 6, adhesiveness between the permeation-limiting layer 6 and the underlying layer (for example, the immobilized enzyme layer 4) can be made good, and fluctuation in performance due to detachment between the immobilized enzyme layer 4 and the permeation-limiting layer 6 can be prevented, which may provide a high performance enzyme electrode with good production stability. Furthermore, a manufacturing process for an enzyme electrode according to the present invention can be applied to production of the enzyme electrode according to the first aspect of the present invention so that even when using a wafer process, a high-quality enzyme electrode can be produced with higher productivity and yield in comparison with the prior art.

In addition, in the present invention, as an enzyme electrode according to the second aspect of the present invention has such a constitution in which, over an immobilized enzyme layer 4, a permeation-limiting layer 6 comprising a fluorine-containing polymer as a main component is placed on its uppermost surface and its surface is formed in such a highly controlled shape having a number of grooves built in on its surface or appropriate surface roughness for its surface; and thereby the permeation-limiting layer 6, of which surface shape is highly controlled, possesses excellent selective permeability, it can provide an enzyme electrode which can be used under wide ranges of the conditions, exhibits good durability during long-term use and also gives higher productivity. In particular, to the step of forming the permeation-limiting layer 6 whose surface shape is highly controlled, a manufacturing process for an enzyme electrode according to the present invention employing a wafer process where a solution comprising a fluorine-containing polymer having a particular structure is applied by spin coating on a wafer and then the applied film is dried to form the permeation-limiting layer 6 is applied, so that the enzyme electrode having such a structure according to the second aspect of the present invention which consistently exhibits desired performance can be produced with higher productivity and yield.

1. An enzyme electrode comprising:
a portion of electrode formed on an insulating substrate;
an immobilized enzyme layer formed over the portion of electrode;
an adhesion layer comprising a silane-containing compound formed over the immobilized enzyme layer; and
a permeation-limiting layer comprising a fluorine-containing polymer having a structure where a pendant group containing at least a fluoroalkylene block is attached to an unfluorinated vinyl-based polymer, which is formed on the adhesion layer.

2. An enzyme electrode as claimed in claim 1 wherein said adhesion layer is a layer composed essentially of a silane coupling agent.

3. An enzyme electrode as claimed in claim 1 or 2 wherein said fluorine-containing polymer is a fluoroalcohol ester of a polycarboxylic acid (A) in which the polycarboxylic acid (A) is contained as the unfluorinated vinyl-based polymer thereof.

4. An enzyme electrode as claimed in claim 1 or 2 wherein said fluorine-containing polymer is a mixture that contains a fluoroalcohol ester of a polycarboxylic acid (A) in which the polycarboxylic acid (A) is contained as the unfluorinated vinyl-based polymer and additionally an alkylalcohol ester of a polycarboxylic acid (B).

5. An enzyme electrode as claimed in claim 4 wherein said fluorine-containing polymer is a copolymer composed of said fluoroalcohol ester of the polycarboxylic acid (A) and the alkylalcohol ester of the polycarboxylic acid (B).

6. An enzyme electrode as claimed in claim 4 or 5 wherein said polycarboxylic acid (B) is selected from polymethacrylic acid, polyacrylic acid or a copolymer of acrylic acid and methacrylic acid.

7. An enzyme electrode as claimed in any one of claims 3 to 6 wherein said polycarboxylic acid (A) is selected from polymethacrylic acid, polyacrylic acid or a copolymer of acrylic acid and methacrylic acid.

8. An enzyme electrode comprising:
a portion of electrode formed on an insulating substrate;
an electrode protective layer covering the portion of electrode;
a binding layer comprising a silane-containing compound, which is formed on the electrode protective layer;
an ion-exchange resin film layer formed on the binding layer;
an immobilized enzyme layer formed on the ion-exchange resin film layer;
an adhesion layer comprising a silane-containing compound, which is formed on the immobilized enzyme layer; and
a permeation-limiting layer comprising a fluorine-containing polymer having a structure where a pendant group containing at least a fluoroalkylene block is attached to an unfluorinated vinyl-based polymer, which is formed on the adhesion layer.

9. An enzyme electrode as claimed in claim 8 wherein said electrode protective layer is made essentially of a urea compound.

10. An enzyme electrode as claimed in claim 8 wherein said binding layer and said adhesion layer are layers composed essentially of a silane coupling agent.

11. An enzyme electrode as claimed in claim 8 wherein said fluorine-containing polymer is a fluoroalcohol ester of a polycarboxylic acid (A) in which the polycarboxylic acid (A) is contained as the unfluorinated vinyl-based polymer thereof.

12. An enzyme electrode as claimed in claim 8 wherein said fluorine-containing polymer is a mixture that contains the fluoroalcohol ester of the polycarboxylic acid (A) in which the polycarboxylic acid (A) is contained as the unfluorinated vinyl-based polymer and additionally an alkylalcohol ester of a polycarboxylic acid (B).

13. An enzyme electrode as claimed in claim 12 wherein said fluorine-containing polymer is a copolymer composed of said fluoroalcohol ester of the polycarboxylic acid (A) and the alkylalcohol ester of the polycarboxylic acid (B).

14. An enzyme electrode as claimed in claim 12 or 13 wherein said polycarboxylic acid (B) is selected from polymethacrylic acid, polyacrylic acid or a copolymer of acrylic acid and methacrylic acid.

15. The enzyme electrode as claimed in any one of claims 11 to 13 wherein said polycarboxylic acid (A) is selected from polymethacrylic acid, polyacrylic acid or a copolymer of acrylic acid and methacrylic acid.

16. An enzyme electrode comprising:
a portion of electrode formed on an insulating substrate;
an immobilized enzyme layer formed on the portion of electrode; and
a permeation-limiting layer formed over the immobilized enzyme layer and placed on the uppermost surface;
wherein said permeation-limiting layer consists of a film essentially comprising a fluorine-containing polymer, and many grooves are built in on the surface of said permeation-limiting layer consisting of the film essentially comprising a fluorine-containing polymer.

17. An enzyme electrode as claimed in claim 16 wherein an average thickness of said permeation-limiting layer is selected within a range of 0.01 to 1 μm; and the surface of the permeation-limiting layer has an irregular shape having a surface roughness within a range of 0.0001 or more and 1 or less fold of said average thickness of the permeation-limiting layer.

18. An enzyme electrode as claimed in claim 16 wherein said fluorine-containing polymer is a polymer having a structure where a pendant group containing at least a fluoroalkylene block is attached to an unfluorinated vinyl-based polymer.

19. An enzyme electrode as claimed in claim 16 or 18 wherein said fluorine-containing polymer is a fluoroalcohol ester of a polycarboxylic acid (A) in which the polycarboxylic acid (A) is contained as the unfluorinated vinyl-based polymer thereof.

20. An enzyme electrode as claimed in claim 16 or 18 wherein said fluorine-containing polymer is a mixture that contains the fluoroalcohol ester of the polycarboxylic acid (A) in which the polycarboxylic acid (A) is contained as the unfluorinated vinyl-based polymer and additionally an alkylalcohol ester of a polycarboxylic acid (B).

21. An enzyme electrode as claimed in claim 20 wherein said fluorine-containing polymer is a copolymer composed of the fluoroalcohol ester of the polycarboxylic acid (A) and the alkylalcohol ester of the polycarboxylic acid (B).

22. An enzyme electrode as claimed in claim 20 or 21 wherein said polycarboxylic acid (B) is selected from polymethacrylic acid, polyacrylic acid or a copolymer of acrylic acid and methacrylic acid.

23. An enzyme electrode as claimed in any one of claims 19 to 21 wherein said polycarboxylic acid (A) is selected from polymethacrylic acid, polyacrylic acid or a copolymer of acrylic acid and methacrylic acid.

24. A process for manufacturing an enzyme electrode comprising the steps of:
forming an electrode film on the main surface of an insulating substrate and then patterning the electrode film to form a plurality of portions of electrodes;
forming an electrode protective layer covering the electrode surface;
forming a binding layer comprising a silane-containing compound on the main surface of the insulating substrate;
forming an ion-exchange resin film layer on the main surface of the insulating substrate;
applying an liquid containing an enzyme to the main surface of the insulating substrate and then drying the insulating substrate to form an immobilized enzyme layer;
applying a liquid containing a fluorine-containing polymer having a structure where a pendant group comprising at least a fluoroalkylene block is attached to an unfluorinated vinyl-based polymer to the main surface of the insulating substrate by spin coating and then drying the insulating substrate to form the permeation-limiting layer; and
dicing the insulating substrate to give a plurality of enzyme electrodes.

25. An process for manufacturing an enzyme electrode as claimed in claim 24 wherein the process is performed in such manner where after said step of forming the immobilized enzyme layer, further step of applying a liquid comprising a silane-containing compound to the main surface of the insulating substrate and then drying the insulating substrate to form an adhesion layer is carried out, and followed by the step of applying the liquid containing said fluorine-containing polymer to the upper surface of the adhesion layer coating t he main surface of the insulating substrate and then drying the insulating substrate to form the permeation-limiting layer,

26. An process for manufacturing an enzyme electrode as claimed in claim 24 or 25 wherein said permeation-limiting layer is a layer being formed by spin coating.

27. An process for manufacturing an enzyme electrode as claimed in claim 25 wherein said silane-containing compound used for forming the adhesion layer is a silane coupling agent.

28. A biosensor comprising an enzyme electrode as claimed in any one of claims 1 to 23.

29. An process for manufacturing an enzyme electrode as claimed in claim 24 or 25 wherein said fluorine-containing polymer is a polymer having a structure where a pendant group containing at least a fluoroalkylene block is attached to an unfluorinated vinyl-based polymer.

30. An process for manufacturing an enzyme electrode as claimed in any one of claims 24, 25 and 29 wherein said fluorine-containing polymer is a fluoroalcohol ester of a polycarboxylic acid (A) in which the polycarboxylic acid (A) is contained as the unfluorinated vinyl-based polymer thereof.

31. An process for manufacturing an enzyme electrode as claimed in any one of claims 24, 25, 29 and 30 wherein said fluorine-containing polymer is a mixture that contains the fluoroalcohol ester of the polycarboxylic acid (A) in which the polycarboxylic acid (A) is contained as the unfluorinated vinyl-based polymer and additionally an alkylalcohol ester of a polycarboxylic acid (B).

32. An process for manufacturing an enzyme electrode as claimed in claim 32 wherein said fluorine-containing polymer is a copolymer of said fluoroalcohol ester of the polycarboxylic acid (A) and the alkylalcohol ester of the polycarboxylic acid (B).

33. An process for manufacturing an enzyme electrode as claimed in claim 31 or 32 wherein said polycarboxylic acid (B) is selected polymethacrylic acid, polyacrylic acid or a copolymer of acrylic acid and methacrylic acid.

34. An process for manufacturing an enzyme electrode as claimed in any one of claims 30 to 32 wherein said polycarboxylic acid (A) is selected from polymethacrylic acid, polyacrylic acid or a copolymer of acrylic acid and methacrylic acid.
